(19) United States
(12) Patent Application Publication
(10) Pub. No.: US 2007/0135838 A1
(43) Pub. Date: Jun. 14, 2007

(54) Safety suture needle assemblies and methods

(76) Inventor: Matthew Earl Meyer, Bountiful, UT (US)

Correspondence Address: RANDALL B. BATEMAN;BATEMAN IP LAW GROUP, 8 EAST BROADWAY, SUITE 550, PO BOX 1319, SALT LAKE CITY, UT, 84110, US

(21) Appl. No.: 10/588,691

(22) PCT Filed: Feb. 9, 2005
(86) PCT No.: PCT/US05/04275

§ 371 Date: Aug. 8, 2006

Related U.S. Application Data
(60) Provisional application No. 60/542,966, filed on Feb. 9, 2004.

(51) Int. Cl. A61B 17/06

(52) U.S. Cl. 606/222

(57) ABSTRACT

Suture needles are a significant safety hazard for medical workers, mainly because of accidental needlestick injuries. Disclosed herein are safety suture needle assemblies and related methods. In particular, variations of a safety suture needle having an activatable sheath or flexible extension member are disclosed. The implementation of certain embodiments of the disclosed invention can result in a desired improvement in safety, including the prevention of unwanted needle punctures, while achieving efficient suturing.

### CROSS-REFERENCES TO RELATED APPLICATIONS

This application claims the benefit of U.S. Provisional Application No. 60/542966, filed Feb. 9, 2004, which is incorporated herein by reference in entirety.

### STATEMENT ON FEDERALLY SPONSORED RESEARCH OR DEVELOPMENT

Not applicable

### BACKGROUND OF THE INVENTION

Surgical needles have been used for centuries for human and veterinary medical purposes such as to close biological tissue that has been separated either by trauma or surgical procedure. The surgical needle is used to penetrate tissue for the advancement of a suture material in order to approximate the separated tissue, in order for the natural healing processes to occur. The surgical needle itself typically has a sharpened end, with various sharp tip configurations for the desired effect or particular tissue. The conventional needle body is made of mainly high strength stainless steel and is formed to many dimensions and shapes. There are various ways for attaching the suture material such as gluing, crimping, swaging, and utilizing shrink wrap type material. The needle is sterilized and packaged to reduce the chance of transmission of infection to the surgical or wound site.

The standard surgical suture needle poses a significantly dangerous hazard to personnel, the patient, and the surrounding patient environment by increasing risks including potential and actual occurrences of accidental puncture. Because of the size of the needle, it is often hard to monitor during surgical procedures. The sharp needle point is particularly difficult to visualize from an end-on perspective. This difficulty can be compounded by poor lighting or the confounding presence of low-contrasting body fluids or foreign matter. The fact that the needle is freely handled, manipulated, and positioned at difficult and dangerous angles in reference to its sharp tip also contribute to the hazard level. Furthermore, considerable forces are often applied to the needle at various points during a surgical procedure, amplifying the consequences of accidental events.

Immediately upon introduction into a sterile surgical environment, the surgical needle is a hazard primarily due to the presence of its sharp exposed tip. In attempts to address the risks of puncture, a variety of devices such as trays, magnetic holders, and cushioned beds for the needle have been introduced for use in procedures. These attempts, however, are not always utilized because of factors including the fast-paced and sometimes chaotic surgical environment in addition to inherent limitations on their efficacy. The frequent resting or storing of needles in unsafe locations during or following a medical procedure poses a hazard that holding devices simply cannot adequately address.

There have been prior attempts at designing a safer suture needle. For example, see U.S. Pat. No. 5,236,443 to Sontag (1993) and U.S. Pat. No. 6,159,233 to Matsuzawa (2000). In Sontag, the needle utilizes an arch-shaped hump that projects upwards from the top middle portion of the needle body. When this needle is held in a needle holder, the hump is depressed to allow protrusion of a sharp tip from what is otherwise a blunted advancing end. Alternatively, Sontag teaches the use of a sliding pin to advance a sharp pointed end. For either the hump or pin approach, one disadvantage is that the outwardly projecting hump or pin may exert unwanted additional dilation upon the tissue that is being penetrated. A second disadvantage is that the hollow distal end that is supposedly blunted while the sharp tip is retracted can still pose a puncture hazard similar to that of a hypodermic needle.

The Matsuzawa patent utilizes a surgical needle type device with a blunted projection tip operating on principles comparable to an electrocautery unit. As the needle device contacts tissue, a high frequency current passes through the device, thereby applying high frequency current to the tissue and achieving local tissue destruction and disruption to effect penetration/incision of the blunt tip of the needle. This approach has disadvantages. First, the requirement, maintenance, or manipulation of an extensive electrical apparatus comparable to an electrocautery unit may not be conducive to the realistic situations of minor laceration repair in a hospital emergency room or urgent care facility or within field operations during training exercises or military operations. Second, there may be less than ideal effects for particularly delicate tissues due to the expected destructive action. Third, the complexity of the device may require considerable training and experience to actually realize a desired minimal level of tissue destruction.

A general difficulty of suture needles with blunted tips is that their utilization may be better for closure of certain tissues such as muscle or particular fascia and not more widely applicable to other tissues. When applied to closing tissue of a denser consistency, the force that is exerted to advance a blunt needle is greater than that needed for a sharper instrument. This greater force becomes an additional hazard to the medical personal and/or patient; for example, such force can induce excessively unnecessary trauma to the tissue being treated. The greater force can also lead to tissue penetration more abruptly than expected and increase the risk of inadvertent needlestick injury to operating personnel.

Despite attempts to address problems such as suture needle stick injuries, there has not been enough practical improvement. Recent government activity acknowledges the persistent problem of such injuries. Since 1998, at least 21 U.S. states have passed needlestick prevention legislation. It is highly desirable to strive for ways to achieve lower incidence of harmful events and the related cost. There is a significant need for the development of safe and effective devices and methods relating to suture needles.

### SUMMARY OF THE INVENTION

In general the terms and phrases used herein have their art-recognized meaning, which can be found by reference to standard texts, journal references and contexts known to those skilled in the art. The following definitions are provided to clarify their specific use in the context of the invention.

When used herein, the term “activator” refers to a mechanical element capable of regulating another mechanical element. For example, an activator or activation mechanism can act on a connected extension shaft and/or sheath so as to change the ultimate position or configuration of the sheath. In another example, an activator can be unitary with respect to a sheath or a sheath and extension.

When used herein, the term “initiator” refers to a component capable of inducing or triggering another device element. For example, an initiator or initiating mechanism can act on an activator to induce activator function. In a more detailed example, energy input, e.g. in the form of heat or electricity, serves as an initiator which in turn influences or alters a property of an activator, which can further lead to an action such as a retracted sheath in the form of a loop flipping out to assume a protective position with respect to a sharp aspect of a sharp object, such as over a sharp end of a needle.

When used herein, the term “sheath” or “sheathing body” or “sheathing assembly” refers to a shield, guard, deflector, or other extending, projecting, or protective piece that is capable of at least partially obstructing access to an item or portion of an item such as the sharp tip of a suture needle. The term can encompass a sheath mechanism. The term can include a sheath that is unitary with a sheath extension mechanism or projection mechanism.

When used herein, the term “blunting mechanism” refers to a component or means that reduces the sharpness of an initially sharp needle point or tip. In an embodiment, the blunting mechanism can be an object with at least one blunt end such as a rod, bar, wire, or means for blunting. In a particular embodiment, the term can be coextensive with a sheath.

When used herein, the term “shape memory alloy” refers to an alloy material capable of undergoing substantial plastic deformation and capable of being induced to return to a substantially original shape. The triggering or inducing can occur by the introduction of energy such as in an initial form of heat or electrical energy. The term refers to a reversible solid-state phase transformation from austenite to martensite on cooling (or by deformation) and the reverse transformation from martensite to austenite on heating (or upon release of deformation). In an embodiment, the shape memory alloy is a binary, ternary, quaternary, or higher order alloy. In an embodiment, a shape memory alloy type is Titanium-palladium-nickel, Nickel-titanium-copper, Gold-cadmium, Iron-zinc-copper-aluminium, Titanium-niobium-aluminium, Uranium-niobium, Hafnium-titanium-nickel, Iron-manganese-silicon, Nickel-titanium, Nickel-iron-zinc-aluminium, Copper-aluminium-iron, Titanium-niobium, Zirconium-copper-zinc, Nickel-zirconium-titanium, or other alloy. In a preferred embodiment, a shape memory alloy is nickel-titanium, also referred to as a generic trade name, nitinol. In an example, nitinol can be nickel-titanium filaments that contract when electrically powered or heated.

When used herein, the term “shape memory plastic” or SMP refers to a plastic or polymer material capable of returning to a preformed shape when the material is properly formulated and treated as known in the art. An example of an SMP is a polymer system of shape-memory polymer networks based on oligo-(espison-caprolactone) dimethacrylate as crosslinker and n-butyl acrylate as comonomer (Lendlein et al., PNAS 2001 98: 842-847). The term can also encompass certain elastic memory composites.

When used herein, the term “suture” is broadly intended to encompass any product as known in the art used to close wounds or connect tissue. The term includes any strand of material used to ligate (tie) blood vessels or approximate tissues.

When used herein, the term “armed” generally refers to a state where the sharp aspect or point of a sharp object is exposed and readily available to carry out its desired function (such as penetration of tissue for a suture needle). Analogously, the term “disarmed” generally refers to a state where the sharp aspect is sheathed (including shielded or guarded) or blunted. The states can be either permanent or temporary.

The following abbreviations are applicable. DS, drawing sheet; SMA, shape memory alloy; NiTi, nickel-titanium.

It is recognized that regardless of the ultimate correctness of any mechanistic explanation or hypothesis, an embodiment of the invention can nonetheless be operative and useful.

In an embodiment, the invention provides a suture needle assembly, comprising a suture needle and an activatable sheath. In an embodiment, said sheath is activatable by electricity. In an embodiment, said sheath is activatable by heat.

In an embodiment, the invention provides a suture needle assembly of claim comprising a suture needle and an activatable sheath, wherein said needle comprises a first tissue penetration end and a second end for suture attachment, an external casing and an internal compartment, an outer surface along a longitudinal axis of said external casing, a sheath activator disposed inside said casing and electrically or thermally responsive to said casing, and said activatable sheath operatively connected to said sheath activator; wherein a portion of said sheath is capable of reaching a protective position proximal to said first end of said needle.

In an embodiment, said sheath activator comprises a shape memory alloy component or shape memory material component. In a particular embodiment, said shape memory alloy component is nitinol.

In an embodiment, said sheath further comprises a catch point, notch, or securing means for maintaining said sheath in an activated position upon activation.

In an embodiment of the suture needle assembly, the sheath activator comprises a shape memory plastic component. In an embodiment, the sheath comprises a shape memory alloy or shape memory plastic.

In an embodiment, the invention provides an above suture needle assembly further comprising an initiator of said sheath activator, wherein said activator is capable of receiving energetic exposure from said initiator. In an embodiment, said initiator comprises a source of heat or electricity.

In an embodiment, the external casing comprises a first outer surface portion and a second outer surface portion capable of forming an electrical circuit.

In an embodiment, the activatable sheath and said activator are unitary.

In an embodiment, the invention provides a suture needle assembly as described herein, further comprising an extension shaft connected at a first end to said activator and connected at a second end to said sheath.

In an embodiment, the invention provides a suture needle assembly as described herein with an activatable sheath that is reversibly capable of activation.

In an embodiment, a suture needle assembly with an activatable sheath, and optionally with a reversibly activatable sheath, is provided in an initially armed configuration. A first activation can then change the initial configuration to a disarmed configuration. In another embodiment, a suture needle assembly with an activatable sheath, and optionally with a reversibly activatable sheath, is provided in an initially disarmed configuration. A first activation can then change the initial configuration to an armed configuration.

In an embodiment, an activatable sheath comprises a hood-shaped shield. In an embodiment, an activatable sheath comprises a cylindrical projection. In an embodiment, an activatable sheath comprises a loop.

In an embodiment, the invention provides a suture needle assembly with an activatable sheath wherein said needle comprises a sheath receiving aperture. In a particular embodiment, said sheath receiving aperture is a groove, notch, or means for receiving a sheath.

In an embodiment, an activatable sheath is capable of assuming a retracted position that is substantially flush along a needle outer surface.

The invention provides a suturing apparatus comprising a suture needle assembly as described above and a needle holder.

In an embodiment, the invention provides a suturing apparatus comprising an above suture needle assembly and a modified needle holder; wherein the modified needle holder comprises a first holding tip and a second holding tip, and the first and second holding tips are energetically connected to an energy source; and wherein the first holding tip and second holding tip are capable of contacting a first and a second needle assembly energy contact surface, wherein said contact surfaces are energetically contacted to said activator of said needle assembly, and wherein said holding tips are capable of delivering energy to said contact surfaces. In an embodiment, the energy source is electricity. In an embodiment, the energy source is heat.

In an embodiment, the invention provides a method of reducing a probability of an accidental suture needle puncture event comprising; providing a safety suture needle as disclosed herein, performing a suturing procedure, activating an electrically or thermally responsive sheathing mechanism of said needle so as to move a sheath of said needle into a protective position with respect to a sharp tip of said needle; thereby reducing a probability of an accidental suture needle puncture event.

In an embodiment, the invention provides a method of suturing, comprising providing a safety suture needle as disclosed herein and performing a suturing procedure with said safety suture needle.

In an embodiment, the invention provides a modified needle holder, comprising a needle holder having a first holding tip and a second holding tip, each electrically connected to a power source and capable of delivering electricity. In an embodiment, the modified needle further comprises a control switch or means for circuit regulation.

In an embodiment, the invention provides a suturing kit comprising a suture needle assembly as disclosed herein and suture material. In an embodiment, the invention provides a suturing kit comprising a suture needle assembly as disclosed herein, a needle holder, and suture material. In an embodiment, the invention provides a suturing kit comprising a suture needle assembly as disclosed herein, a modified needle holder, and suture material.

In an embodiment, the invention provides a suture needle comprising a unifying element, wherein the unifying element is either continuously connected to a needle casing or connected at multiple points thereto, wherein said unifying element comprises a shape memory material component and is capable upon a fracturing event of said needle casing of preventing dissociation of a needle part from another needle part or the remainder of the needle body. In an embodiment, the shape memory material component is a shape memory alloy; in a particular embodiment the shape memory alloy is nitinol.

The invention provides a safety suture needle assembly and mechanism with sharp tip point sheath or blunting extension. The invention provides a mechanism of activation for the sheath or blunting extension comprising a Shape Memory Alloy assembly, methods of construction, and variations thereof. The invention provides a safety suture needle mechanism activator in the form of a modified needle holder. The invention provides such an activator in the form of an assembly to be attached to an existing needle holder. Variations on modes of the activation mechanism are also included. The invention provides an optionally reversible mechanism of activation for a sheath or blunting extension.

A problem can exist with suture needle devices in that due to physical properties (e.g. size, material properties, instrument shape, manufacturing processes and/or treatments) and the forces occasionally applied to the devices. For example, conventional needle devices can fail by breaking or snapping under certain conditions. In embodiments of the present invention, the overall device can at least partially facilitate the function of holding external broken pieces of the needle body together. This function can be due to one or more factors including an internal mechanism, design, and/or construction. The function can aid in the prevention of allowing broken pieces to fall or lodge within a particular body tissue and adversely affect the health or comfort of a patient or instrument user. In an embodiment, device components are attached along the longitudinal axis of the needle body, so that if a sufficient force were applied upon the needle approximating a point of mechanical failure, one or more internal components can keep the external body of the needle together so as to prevent the separation of the broken parts from a point of connection to the device.

The invention provides a method of suturing comprising providing a suture needle assembly device, performing a suturing procedure, and activating a suture needle sheath.

The invention provides a method of preventing or reducing the incidence of transmission of body fluid borne pathogens to a patient or a person conducting a medical procedure with a sharp object. In an embodiment, the sharp object is a suture needle. The invention provides a method of preventing or reducing the incidence of unwanted punctures of a sterile surgical environment caused by sharp medical objects.

In an embodiment, the needle itself comprises a Shape Memory Alloy (SMA) sheath mechanism. In an embodiment, the SMA sheath mechanism is capable of activation. In an embodiment, the activation for a SMA mechanism is achieved by ohmic or thermal heating of the SMA material. In an embodiment, the needle itself is at least partially composed of an SMA mechanism.

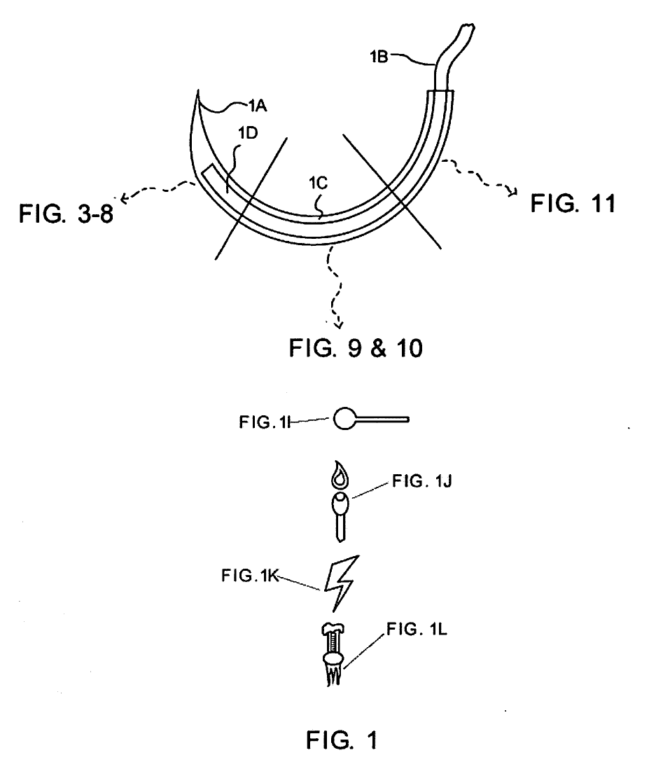

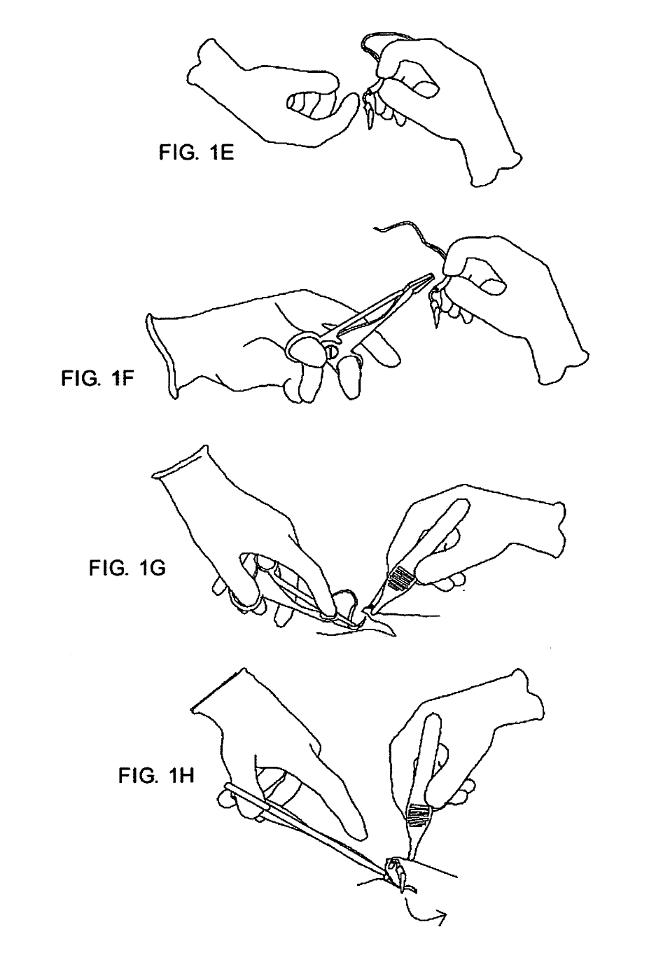

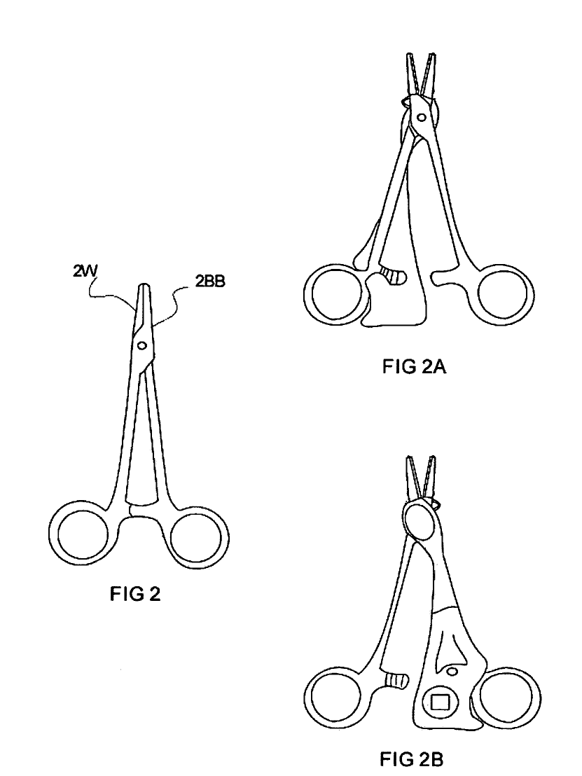

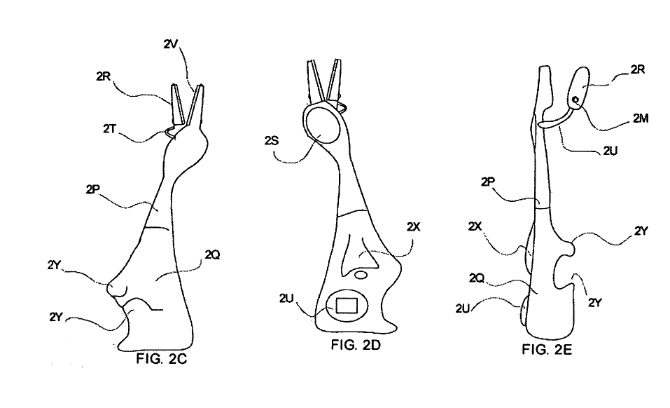

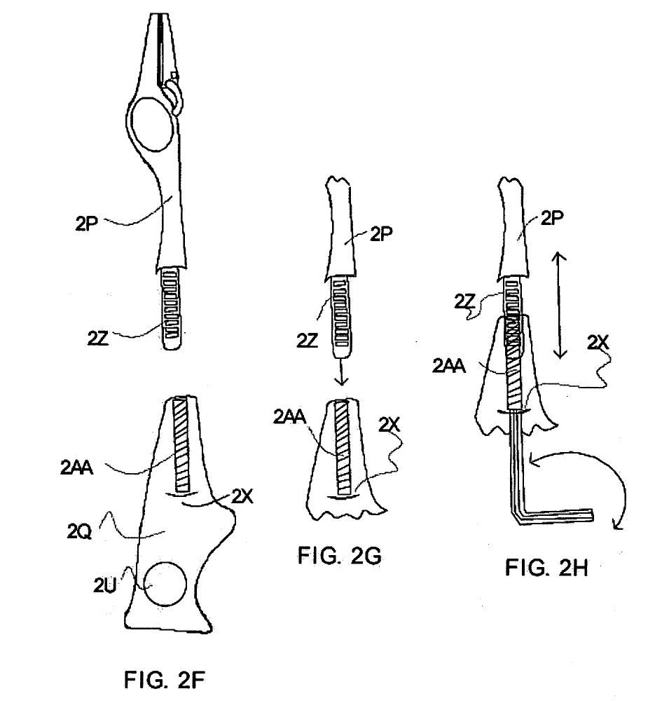

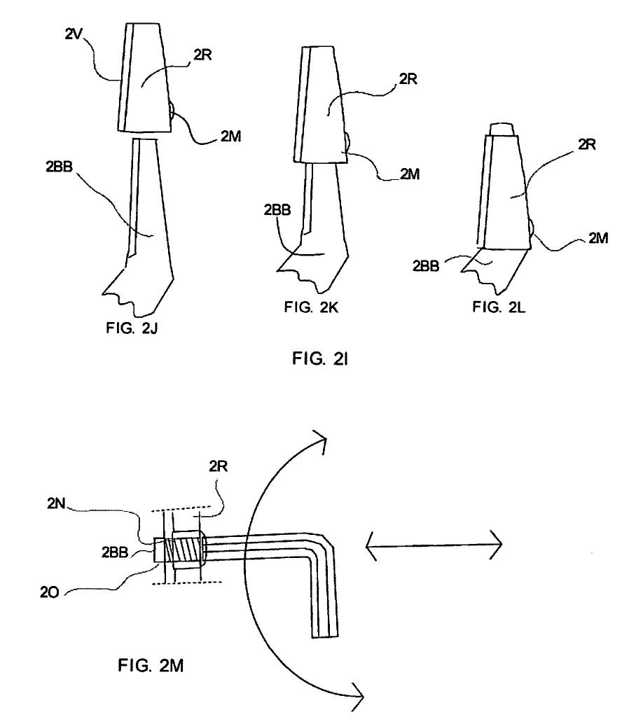

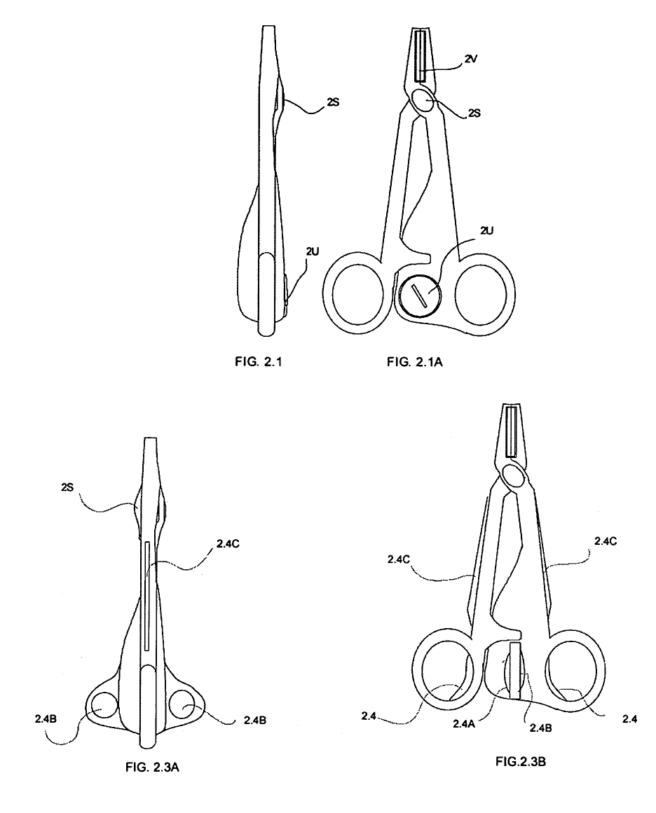

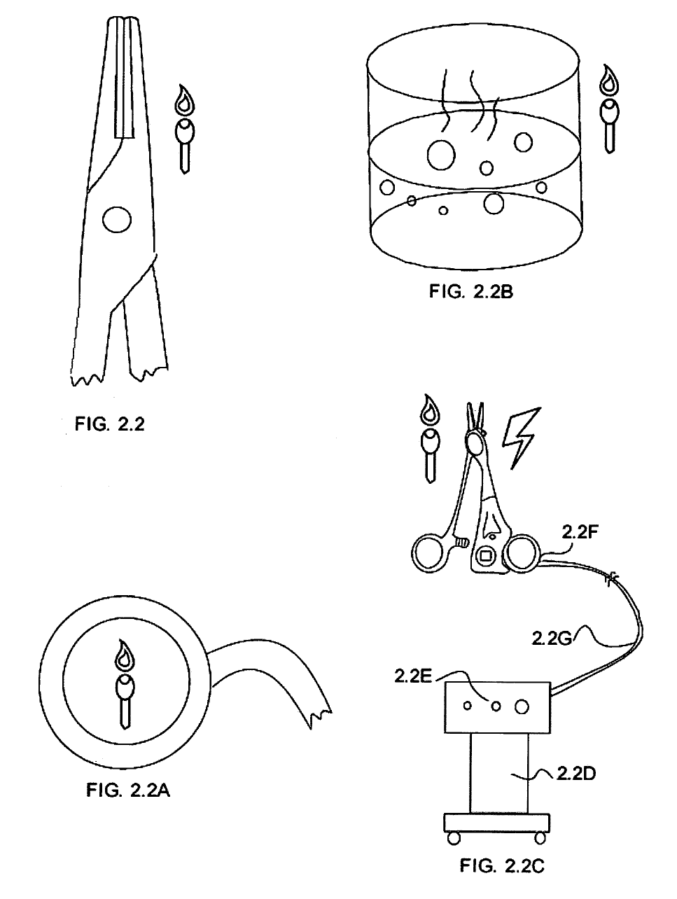

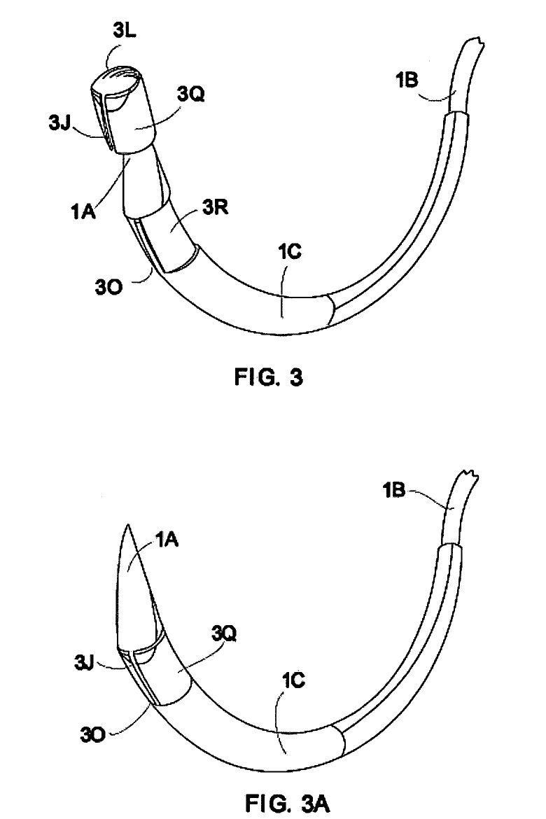

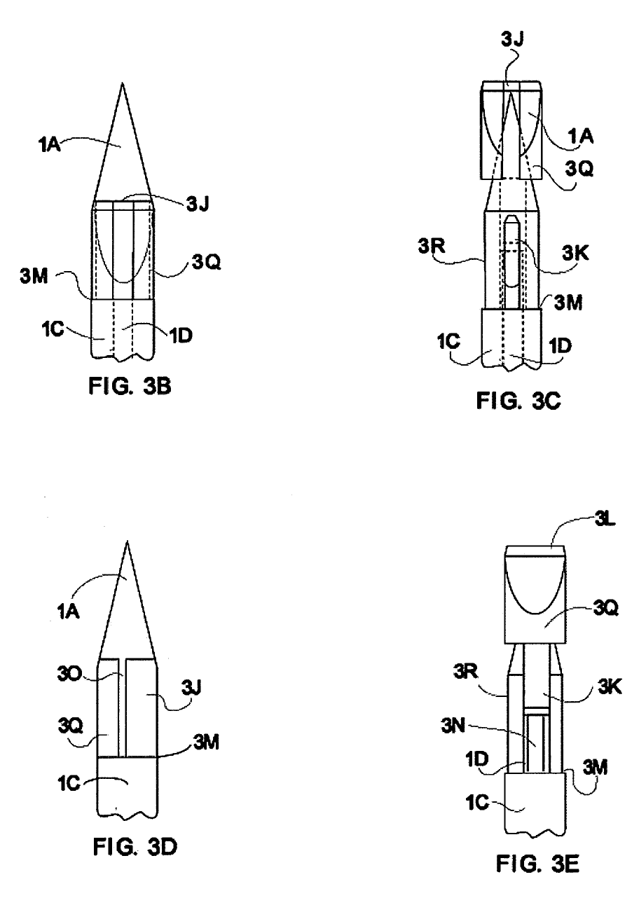

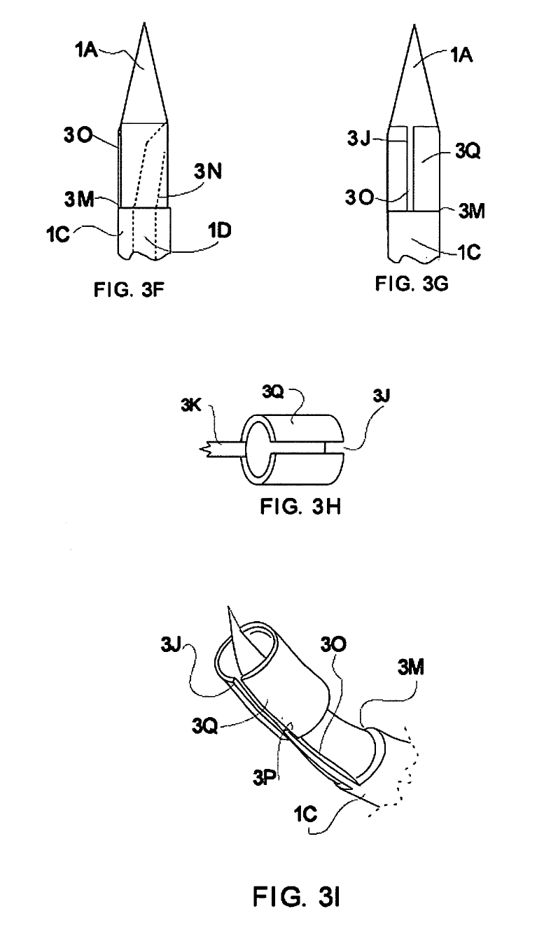

A general figure for an embodiment of the present invention is illustrated in FIG. 1. The body as a whole is designed for the penetration of tissue and utilizes a mechanism for the sheathing/blunting of the sharp distal end (FIG. 3-8). In an embodiment, the sheathing/blunting mechanism is activated by an external source (e.g. FIGS. 2.1A, 2A) by using a Shape Memory Alloy (SMA) material assembly. Several variations of the activation mechanism and SMA material assembly are presented. In a particular embodiment, a variation is selected or preferred due to factors such as the overall small size of the SMA assembly, manufacturing of components, and assembling (fitting together, by hand or machine assistance) of pieces at a small device scale. The variations presented can be applicable and can be adapted or utilized based on manufacturing techniques as known in the art.

In preferred embodiments, there are generally two designs demonstrating utilization of the properties of the SMA mechanism. A useful characteristic of an SMA such as nitinol (nickel-titanium alloy) is to contract when heated either directly or by ohmic heating through the passage of electricity. A second useful property is the ability of the SMA (e.g. nitinol) to return to a preformed shape after treatment such as by heating. The first characteristic gives the opportunity to incorporate a reversible or on/off mechanism into the general design, or an at-rest and contracted state utilized in said mechanism. In an embodiment, the ability of nitinol to deform, for example by contracting from about 6% to about 10% of its overall initial length upon heat treatment, is applicable to a reversible on/off mechanism. In an embodiment, the deformation ability is applicable for an extended/retracted form, with the application of an extension spring. In an embodiment, after the heating process and when the Nitinol cools, the extension spring can apply force to stretch the nitinol towards its original elongated shape.

The second characteristic of nitinol to return to a preformed shape is applicable to a mechanism of activation with an operator permanently or continuously activating the assembly. In an embodiment, activating the assembly allows the preformed wire to move from a deformed shape to its original shape. In an embodiment, the nitinol assembly returns to a preformed elongated form, thereby linearly extending the said blunting/sheathing mechanism in order to cover and/or guard the sharp point of the suture needle. The contraction of an SMA assembly through ohmic heating can be achieved by the administration of electrical current through a needle holder. Examples of a needle holder and variations are shown in FIG. 2.

In an embodiment, a needle holder comprises two electrical contact tips. A first electrical contact tip is routed and appropriately insulated to serve as a positive terminal. A second electrical contact tip is analogously routed and insulated as a negative terminal. In an embodiment, said first and second tips are capable of forming a circuit. In an embodiment, the needle holder can make contact with a suture needle of the invention, for example corresponding to a positive needle contact point or side and a negative needle contact point or side. The needle is integrated with a sheathing/blunting mechanism and activation mechanism. Thus when the needle holder is in operative (e.g., electrically conductive) contact with the needle, the circuit is capable of delivering power to the activation mechanism. In an embodiment, transmission of electrical current to the SMA results in the desired characteristic of contraction or shape memory return. In a particular embodiment, the sheathing blunting mechanism and activation mechanism are unitary.

In an embodiment, the invention provides devices and methods designed to prevent the unwanted puncture or penetration of tissue and/or material (e.g. draping, gowns, masks, other clothing, etc.) relating to a surgical environment that aspirationally is sterile. The devices and methods, however, are not necessarily confined only to surgical situations but can be useful for other applications. For example, the linear projection of a sheath to obstruct or provide the protection of a blunt barrier near a sharp tip can be utilized for objects other than needles.

For the particular embodiment of suture needle applications, a benefit is to prevent the transmission of body fluid borne pathogens from a patient to a person conducting a medical procedure. The prevention of transmission is manifested through the introduction of a sheathing body which guards the sharp needle tip, thereby protecting a worker from accidental penetrating exposure. The term sheathing body is widely used in this context; certain embodiments for sharp point protection, however, utilize a blunting effect or a deflector type assembly to warn the operator of the said device and of the relation of the operator to the close proximity of the sharp point. For example, an operator can be warned by minimally harmful or non-harmful contact with the sheathing body. Under common conditions the force actually exerted during an accidental puncture is low, such as due to snagging or inadvertently brushing up directly against the sharp point. The deflector type design is expected to significantly aid in preventing the occurrence of an accidental puncture by deflecting contact with the sharp point.

In an embodiment, the needle body described and illustrated herein is of the conventional suture needle design. In a particular embodiment, the needle body has the generally existing round variety. The devices and methods of the invention can be adapted and applied to other needle shapes. The devices and methods of the invention can be modified to accommodate tip variety and/or needle body shape; for example, the basic functional shape and design of the said sheathing mechanisms and mechanisms of activation can be so modified.

The devices and methods of the invention are adaptable to other assemblies capable of being sheathed (e.g. shielded, blunted, etc.) for example by extension or projection near or over a sharp aspect of a sharp object. Embodiments of the invention are therefore widely applicable for precision processes or protection from sharps such as with trocars, razors, hypodermic needles, and the like.

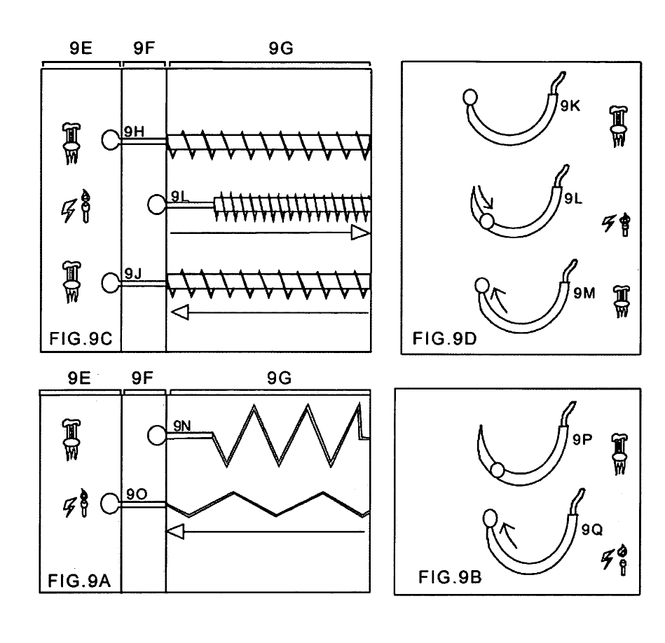

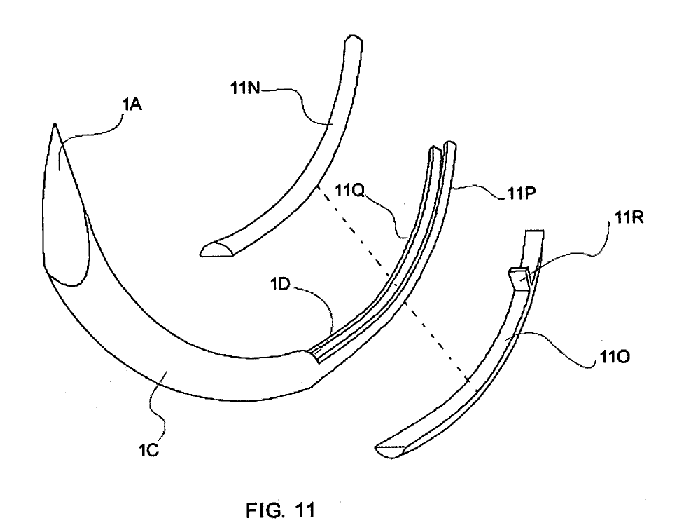

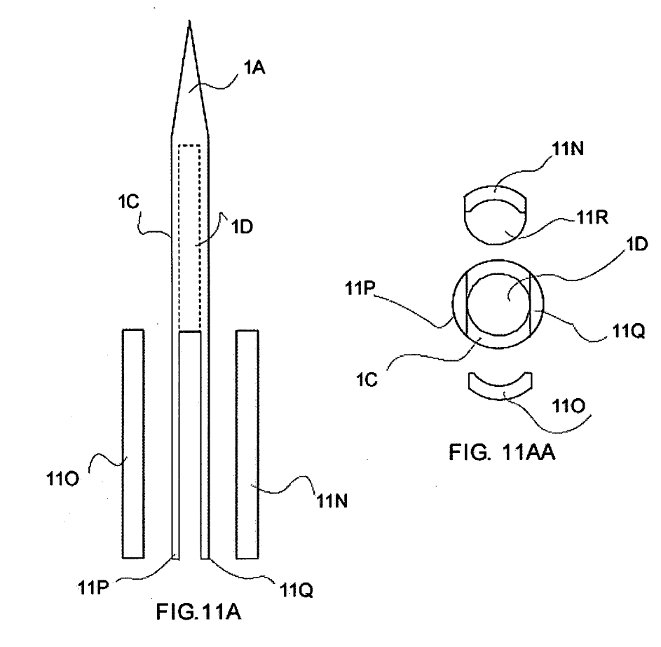

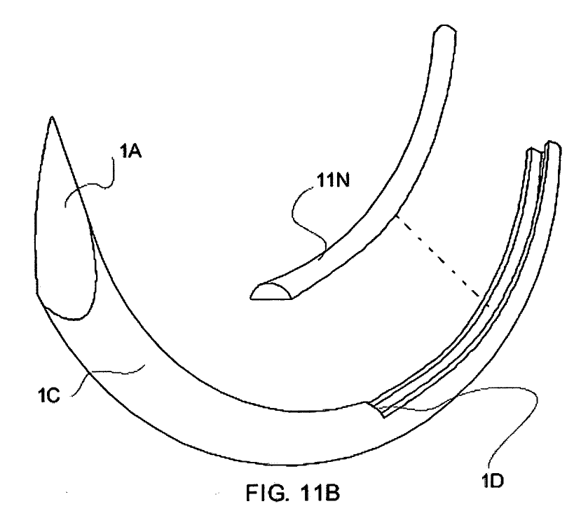

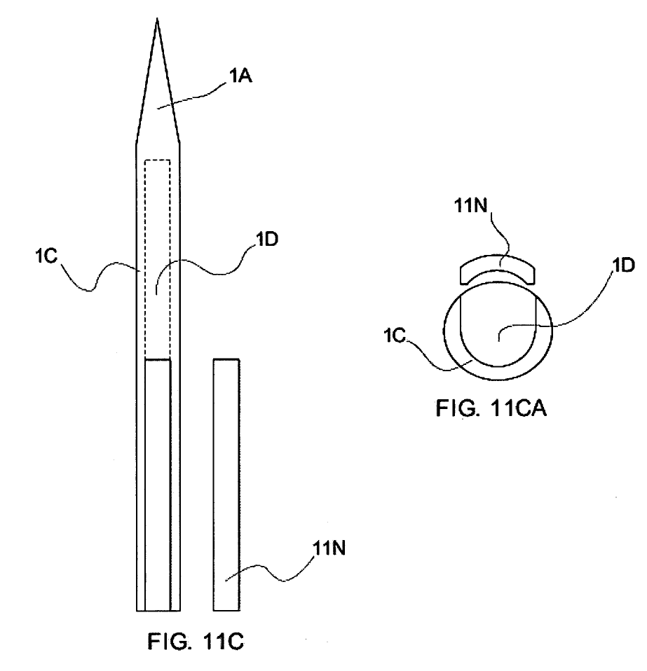

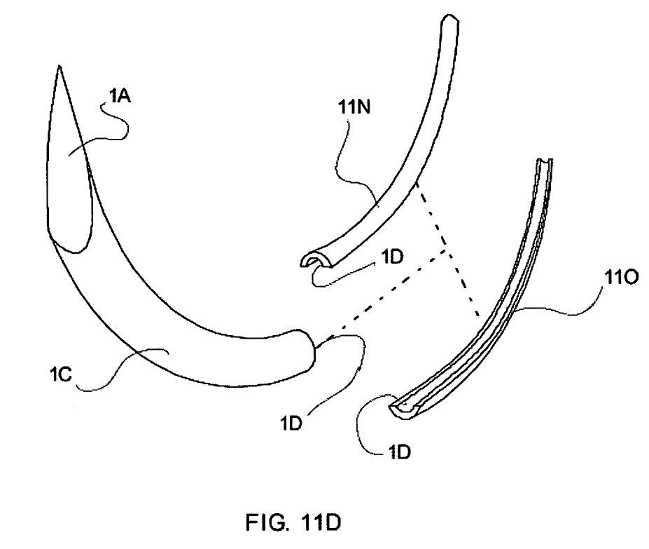

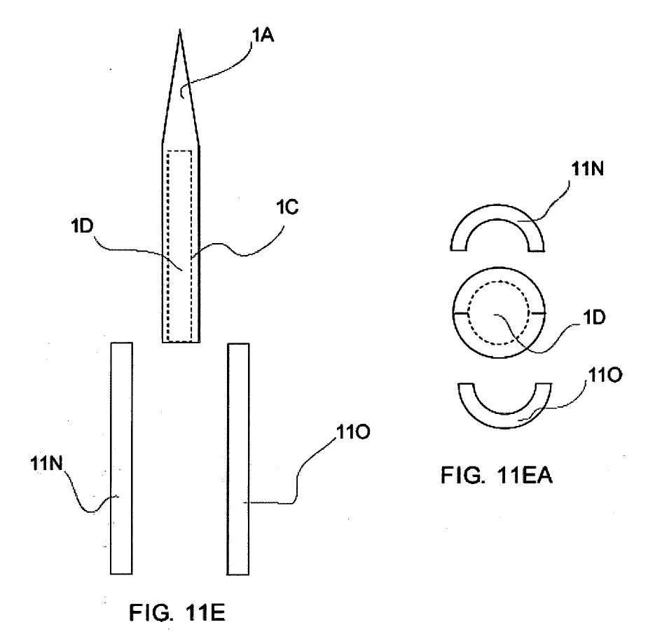

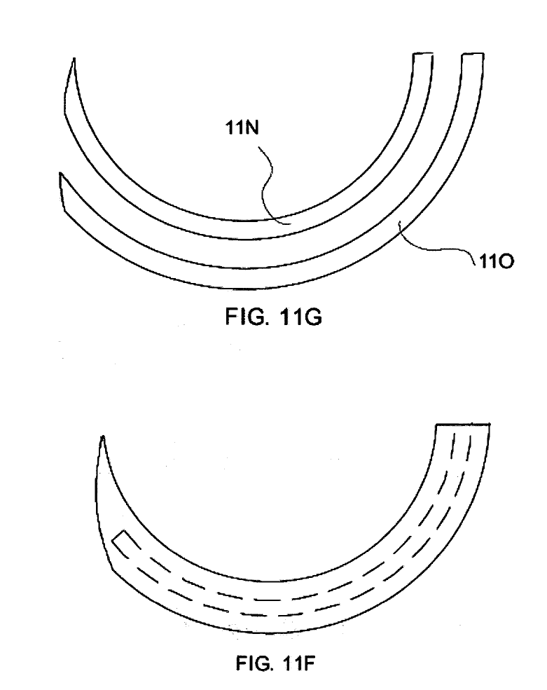

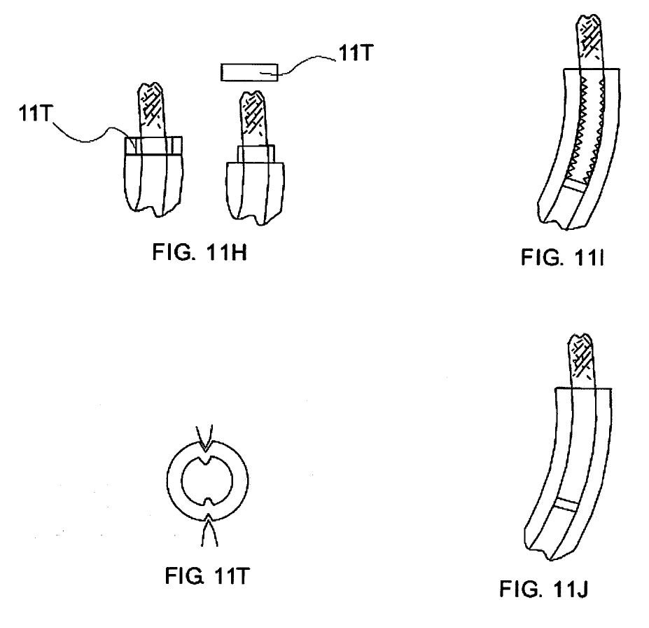

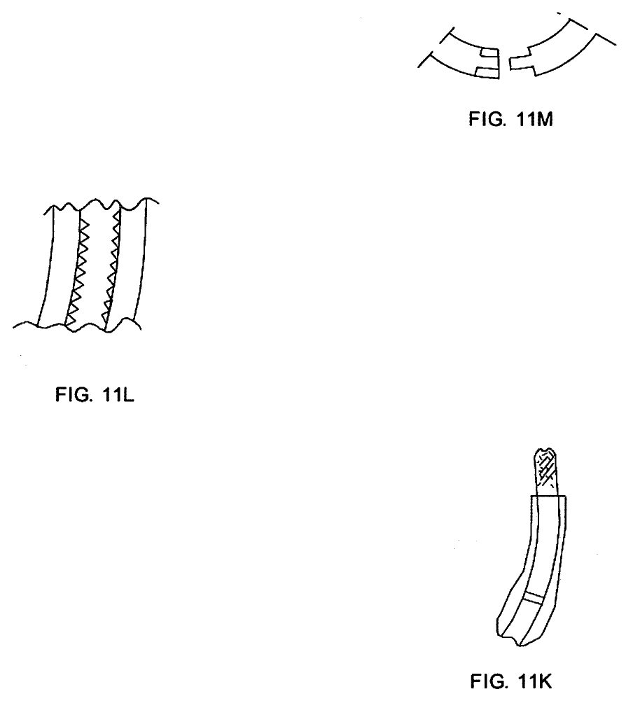

An embodiment of the invention is illustrated in FIG. 1. FIG. 1 shows a general needle body with an inner cannula 1D, an outer main body 1C, a needle tip 1A, and suture material 1B. FIG. 11 is used to denote the sheath blunting or projection of an assembly which is further described in FIGS. 9B and 9D to illustrate an activation mechanism (optionally reversible) and the blunting effect and location of a general sheath for sharp point protection. FIG. 1L represents an example of the cold phase of the shape memory alloy component. FIG. 1K represents the application of electricity to a mechanism of activation (e.g., the shape memory alloy component). FIG. 1J represents the application of heat to the said mechanism of activation. Heat can be applied by exposure to a heat source such as heated water, a hot plate, fire, induction, and other means.

In an embodiment involving a needle holder variation, there can be an integrated delay feature, for example to allow the needle holder to continue to supply energy (e.g. heat or electricity) for a period of time after an operator has released a regulatory mechanism (e.g. a control switch). Such an embodiment is useful to account for the time necessary for the suturing needle to pass through a tissue being sutured when it may be practically difficult or inconvenient for a device operator to maintain continuous contact with a regulatory mechanism. In another embodiment the suture needle assembly can incorporate an opposing force/rebound tension system that allows a slower activation to address the time issue. In another embodiment, an override switch is included in the needle assembly in operative contact with the activation mechanism; when the override switch is in operative contact with the needle holder contact points, the needle assembly is maintained in the activated configuration which can be an armed or disarmed state.

In an embodiment, a basic explanation of the operation is as follows. The needle instrument can be in a first state that is a resting state. In this first state, the default is for the sheath to be in a first position that is a protective position with respect to a sharp tip of the needle. The resting state is in part attributable to the application of a substantially constant first force exerted upon a shape memory alloy component. In an example, the first force is achieved by means of a spring. Upon application of a second force to the shape memory alloy component, the activation mechanism (of which the shape memory alloy component can be integral) is triggered, thereby allowing the sheath to move to a second position which is a non-protective position. In a particular embodiment, the second force is related to energy input from heat or electricity. The application of the second force is able to overcome the still existing effects of the first force and thus maintain the needle in an activated state. By temporary or permanent suspension of application of the second force, the first force is no longer overcome, thereby allowing a return of the needle instrument to a resting state. Certain embodiments of the invention can therefore be reversible.

In an embodiment of the invention, the initial state can be determined to be an activated state with the second state being a resting state. For example, the initially activated state can correspond to the needle being in an armed configuration with the available sheath not providing substantial protection against contact with the tip by a worker. Alternatively, the initial state can correspond to the needle being in a disarmed configuration.

In an embodiment, the sheath activation mechanism (optionally integrated with a sheath extension or projection mechanism) is activated by heating. A heating source can be selected depending on the characteristics of the activation mechanism desired and the relative ability and cost effectiveness of producing such an assembly.

In an embodiment, the present invention is not limited to the use of the material nitinol as the sole material for the actual activation of the needle assembly. Other technology is adaptable for use with devices and methods of the invention and can include micro-pizeo electric actuators, electro active polymers, shape memory plastics, micro-pistons, and other means of micro-linear activation. For example, the activation mechanism or sheath extension/projection mechanism can incorporate one or more of these means.

In particular embodiments of the invention, one or more objects and advantages are achieved. In an embodiment, the invention provides multiple ways to configure or construct a sheathing assembly. These ways can be optionally influenced by an assessment of factors such as desired manufacturing techniques and possibilities, and consumer needs and preferences. In an embodiment, the invention provides multiple ways to configure or construct a mechanism of activation. In an embodiment, the invention provides multiple ways to construct the needle embodiment which can optionally be preferred according to concerns regarding manufacturing means, processes, materials, methods, and consumer preferences. In an embodiment, the invention provides multiple ways for activating the needle body safety suture mechanism.

In preferred embodiments, the invention provides two general forms for the sheathing/blunting assembly to act in a protective manner: (a) to arrive safely disarmed to the consumer with the capacity to be armed and with the said on/off characteristics, or (b) to arrive to the consumer armed, until the point where the operator of the device chooses to disarm the needle, thereby rendering it relatively safer (optionally from that time forward).

In an embodiment, a suture needle assembly has a linear hollow shaft internally disposed along a longitudinal axis of the needle body, and the needle tip itself is constructed to accept or be in operative connection with a sheathing/blunting mechanism. It is recognized that in comparison to a conventional suture needle that is curved, the referenced longitudinal axis for a needle body of the present invention can be a curved/arcuate longitudinal axis. The needle body itself is designed to allow the application or insertion of a mechanism of activation. The mechanism of activation is designed according to desired possible functions. In an embodiment, a method of activation for a linear projection assembly can utilize one or both of two options including ohmic heating and thermal heating. Various factors can determine the optimality for a particular embodiment or application.

In an embodiment, the attachment, assembly, and manufacture/construction of all parts can be performed using knowledge of one of ordinary skill in the art (e.g. regarding welding, crimping, gluing, joining, grinding, drilling, laser cutting, forging, photoelectric construction, fabrication and manipulation of shape memory materials including shape memory alloys and/or shape memory plastics, except where specified. Various molding technologies such as injection molding is applicable for liquid metals and plastics.

In an embodiment, a particular exception can be noted for use of ohmic heating in relation to the needle body for operation of the invention according to its, the needle body is designed and constructed so as to allow the free transmission of electricity to the mechanism of activation. In a preferred embodiment, the design and construction encompasses materials so as to allow the activation mechanism to be electrically insulated. A power source can be located within or externally appended or connected to a needle holder. As would be understood in the art, in an embodiment the power source can contain a pulse width modulation circuit or other control mechanism, e.g. to regulate the amount of current so as to conserve power consumption and also prevent overheating of the activation mechanism (e.g. SMA) assembly. In an embodiment, electrical precautions can also be included to minimize the safety or comfort risk of exposure of a worker or patient to electricity.

In an embodiment, one or more of the following can be unitary and made of nitinol: a sheath, sheath extension shaft (extension), and activator; a sheath and extension; and an extension and activator.

In an embodiment, devices and methods of the invention are readily adaptable to suture needles that have a cross-sectional geometry other than round.

In an embodiment, a needle of the invention is supplied in a preset configuration where the needle is armed and is reversibly activatable to a disarmed configuration. In an embodiment, the preset armed needle is once activatable to the permanently disarmed configuration.

In an embodiment, a needle of the invention is supplied in a preset configuration where the needle is disarmed and is reversibly activatable to an armed configuration. In an embodiment, the preset disarmed needle is once activatable to the permanently armed configuration.

Separate embodiments of the invention are also intended to be encompassed wherein the terms “comprising” or “comprise(s)” or “comprised” are optionally replaced with the terms, analogous in grammar, e.g.; “consisting/consist(s)” or “consisting essentially of/consist(s) essentially of” to thereby describe further embodiments that are not necessarily coextensive.

### BRIEF DESCRIPTION OF THE FIGURES

Drawing Sheet 1 (DS1); FIG. 1 illustrates an overview of a suture needle with references to related figure sets.

DS2; FIG. 9A to FIG. 9G illustrate suture needle activator mechanisms.

DS3; FIG. 1E-1H illustrate usage of a needle assembly device with a break-away tip and a suturing apparatus of a needle holder and needle combination.

DS4; FIG. 3 and FIG. 3A illustrate a needle assembly with a radial/cylindraceous sheath.

DS5; FIG. 3B-FIG. 3E illustrate various views and portions of a needle assembly with radial/cylindraceous sheath.

DS6; FIG. 3F-FIG. 3I illustrate various views and portions of a needle assembly with radial/cylindraceous sheath.

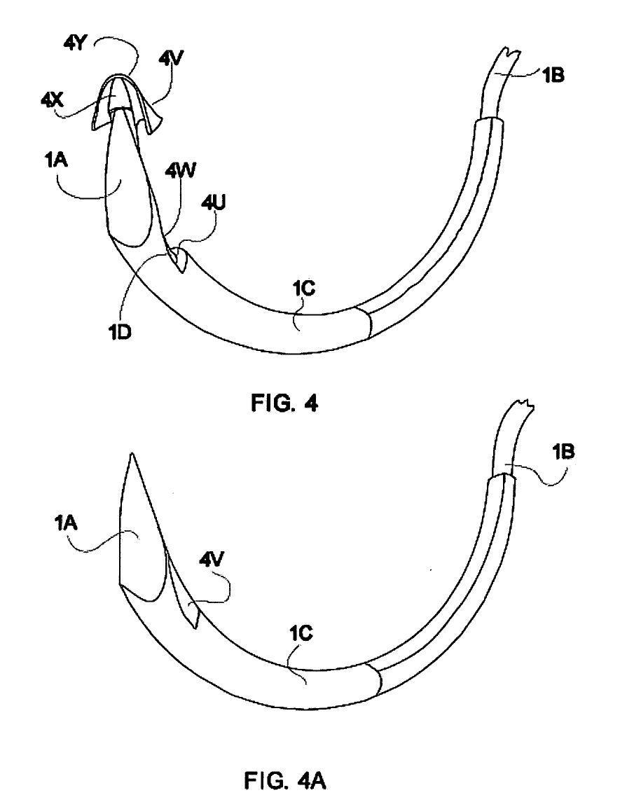

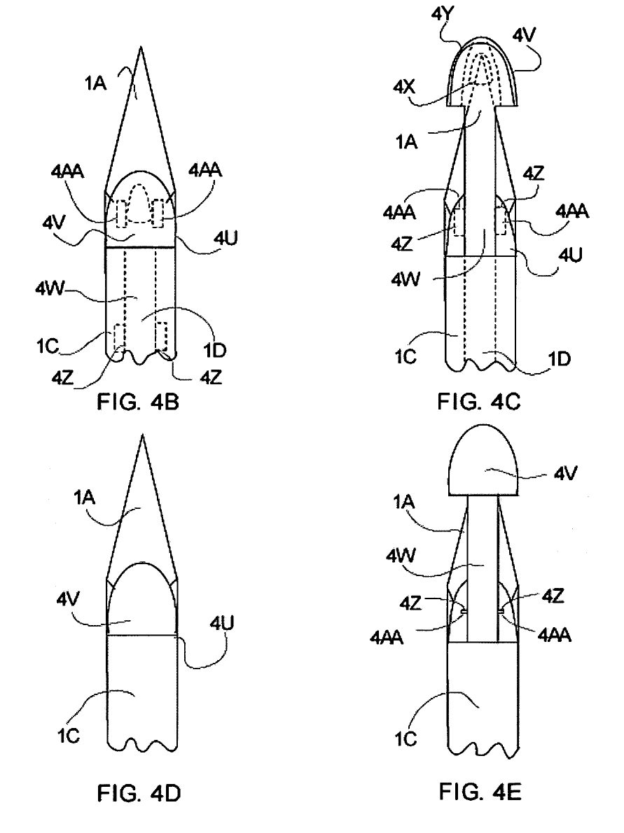

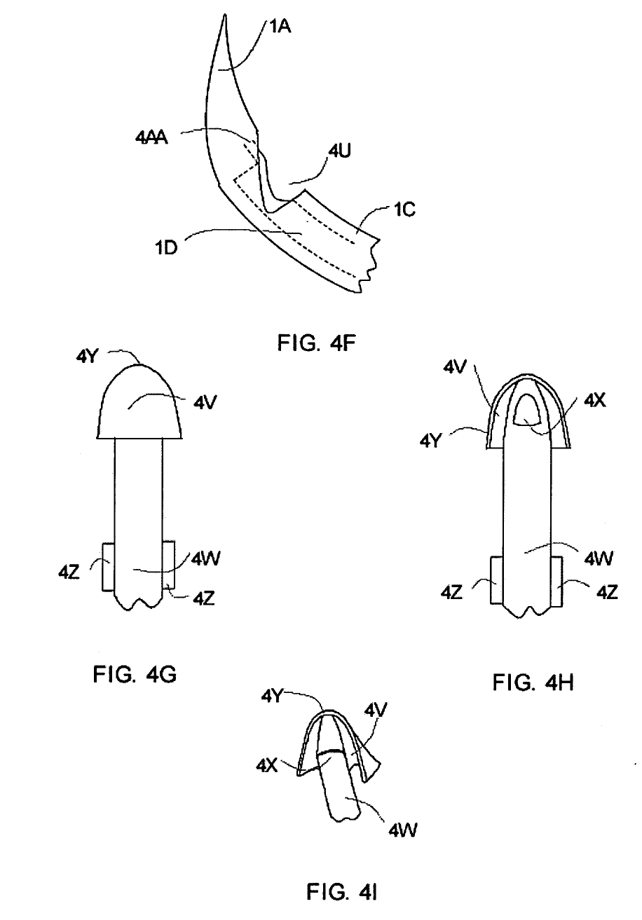

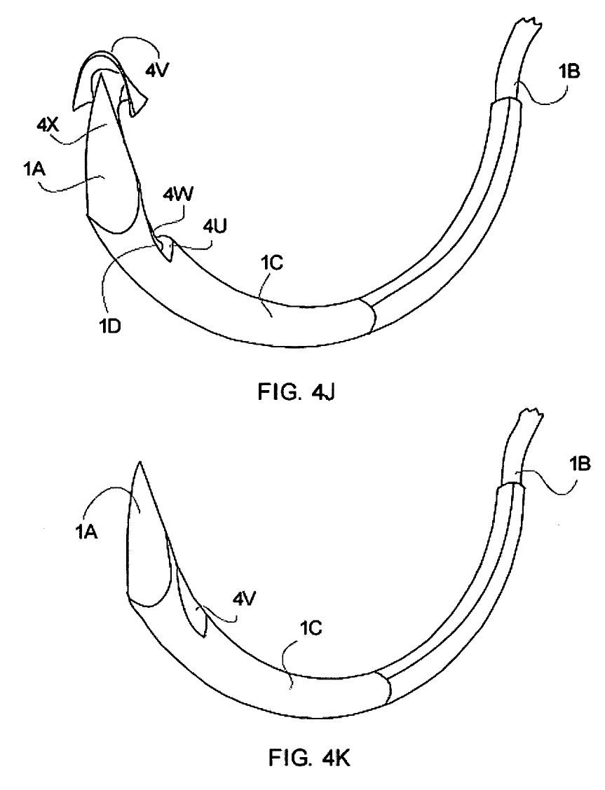

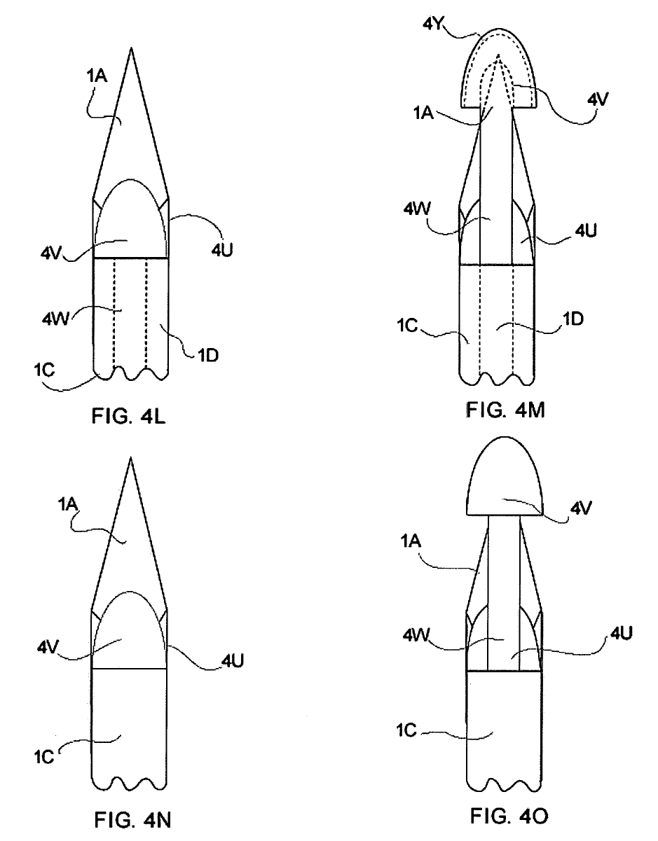

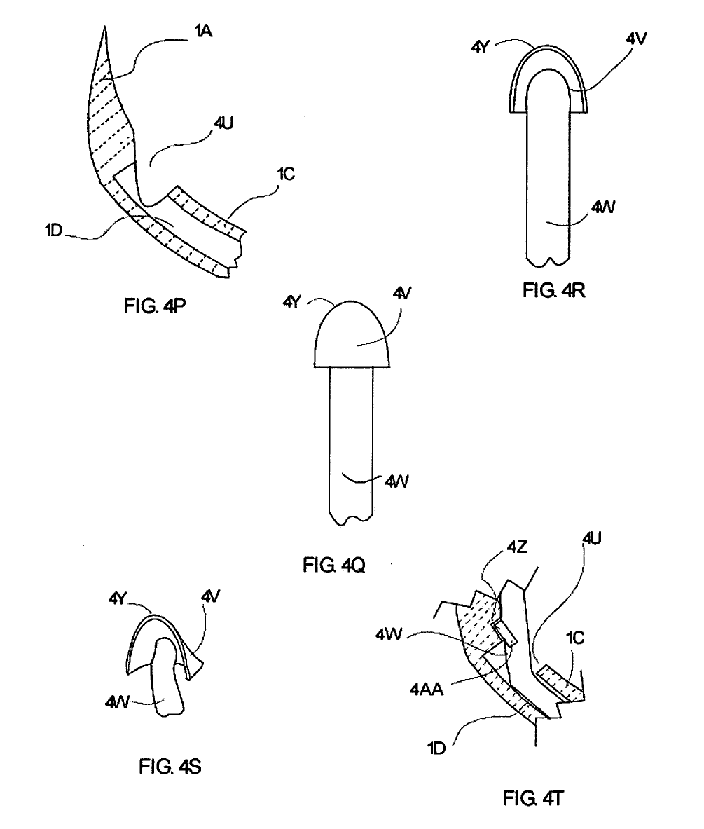

DS7; FIG. 4 and FIG. 4A illustrate a needle assembly with a shield-like sheath.

DS8; FIG. 4B-FIG. 4E illustrate various views and portions of a needle assembly with a shield-like sheath.

DS9; FIG. 4F-FIG. 4I illustrate various views and portions of a needle assembly with a shield-like sheath.

DS10; FIG. 4J-FIG. 4K illustrate various views and portions of a needle assembly with a shield-like sheath.

DS11; FIG. 4L-FIG. 4O illustrate various views and portions of a needle assembly with a shield-like sheath.

DS12; FIG. 4P-FIG. 4T illustrate various views and portions of a needle assembly with a shield-like sheath.

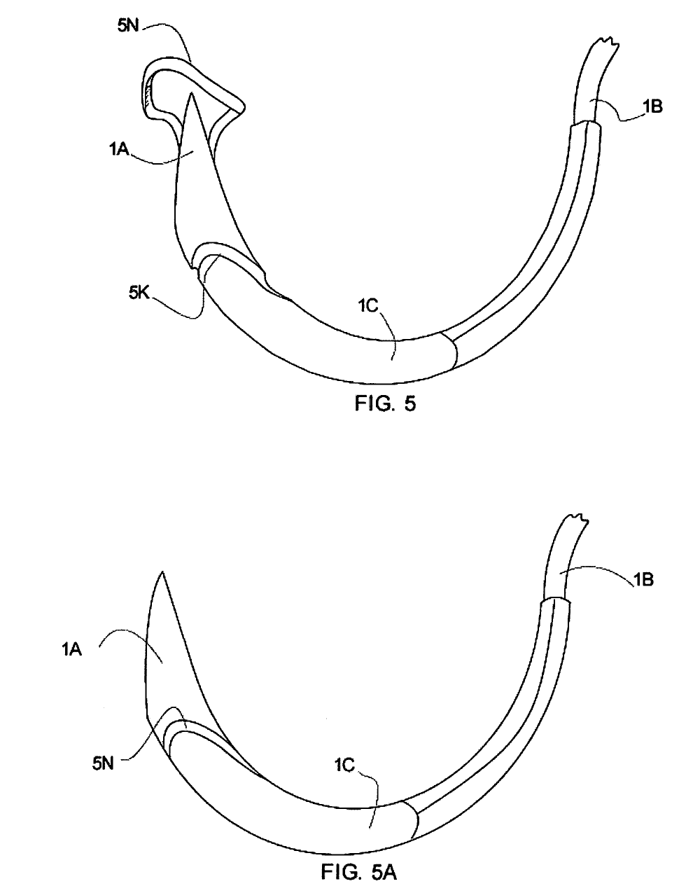

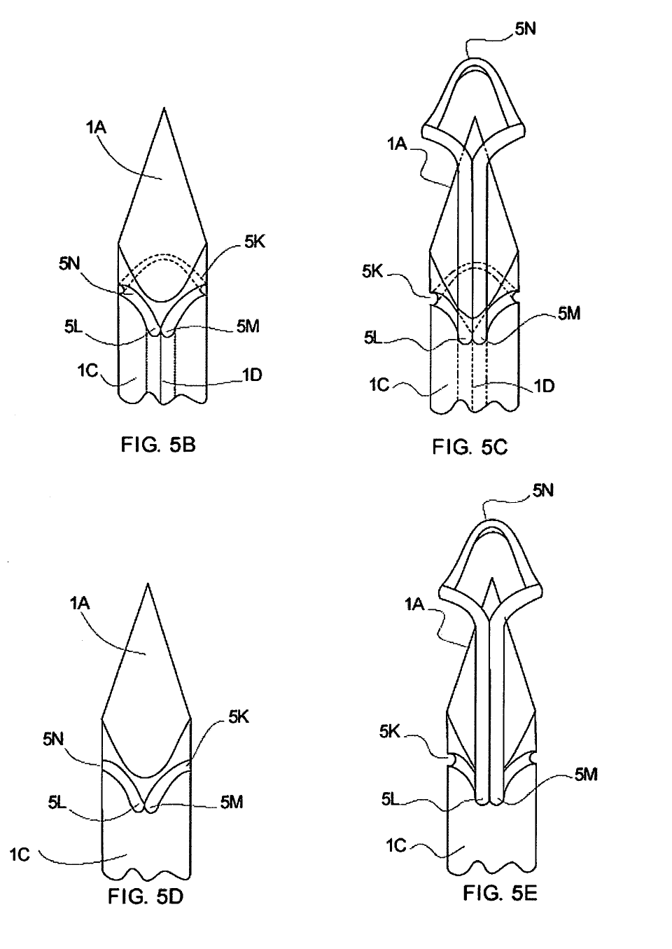

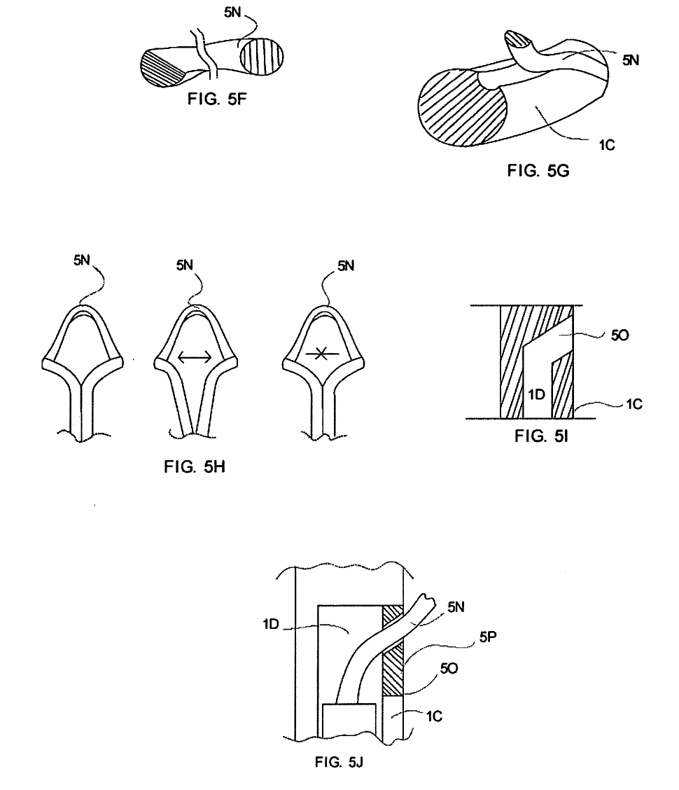

DS13; FIG. 5 and FIG. 5A illustrate a needle assembly with a loop-shaped sheath.

DS14; FIG. 5B-FIG. 5E illustrate various views and portions of a needle assembly with a loop-shaped sheath.

DS15; FIG. 5F-FIG. 5J illustrate various views and portions of a needle assembly with a loop-shaped sheath.

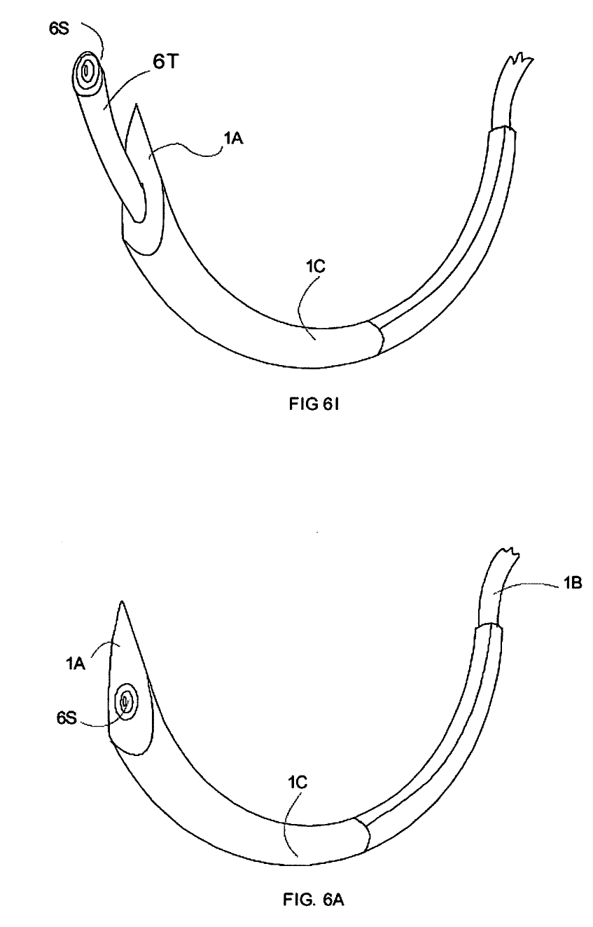

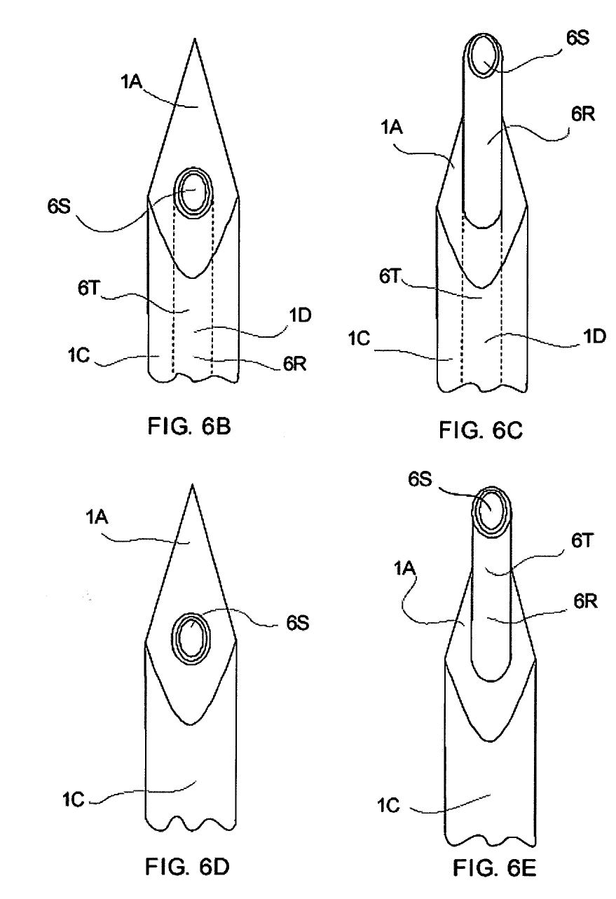

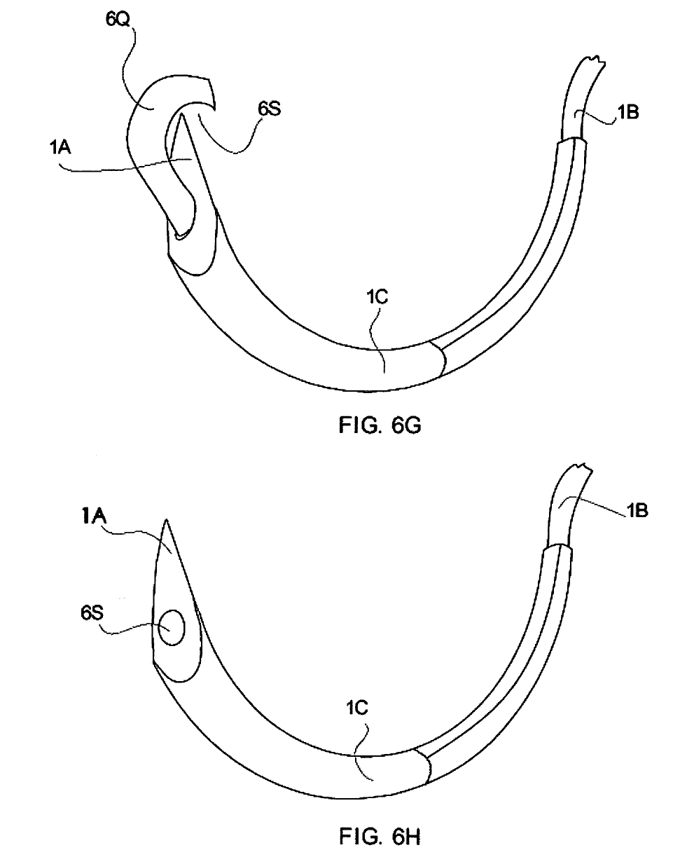

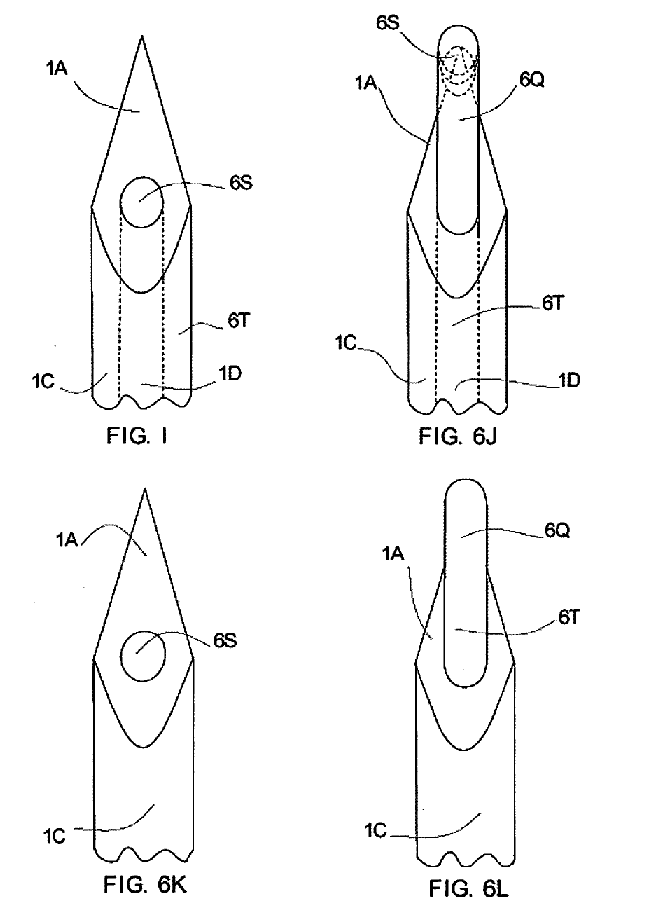

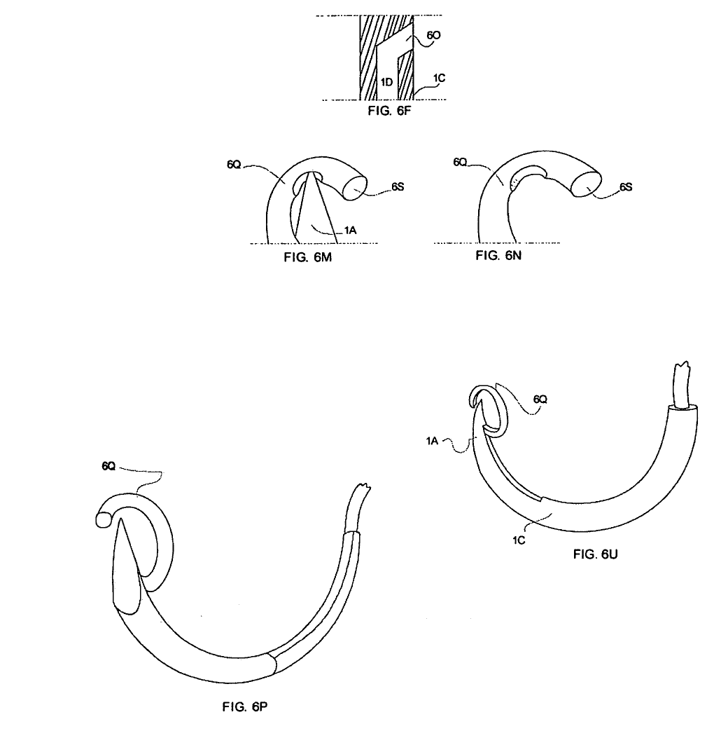

DS16; FIG. 6 and FIG. 6A illustrate a needle assembly with a rod projection blunting system.

DS17; FIG. 6B-FIG. 6E illustrate various views and portions of a needle assembly with a rod projection.

DS18; FIG. 6G-FIG. 6H illustrate various views and portions of a needle assembly with a rod projection.

DS19; FIG. 6I-FIG. 6L illustrate various views and portions of a needle assembly with a rod projection.

DS20; FIG. 6M-FIG. 6U illustrate various views and portions of a needle assembly with a rod projection.

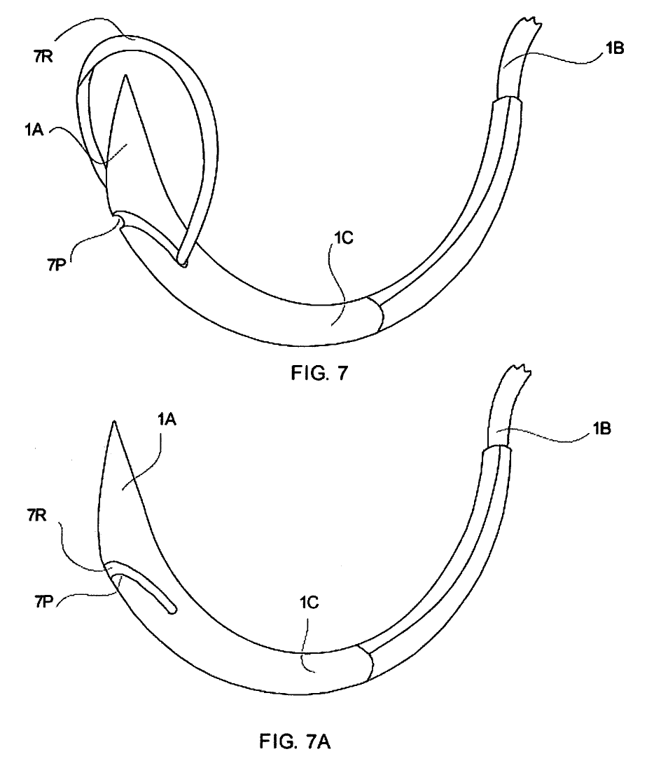

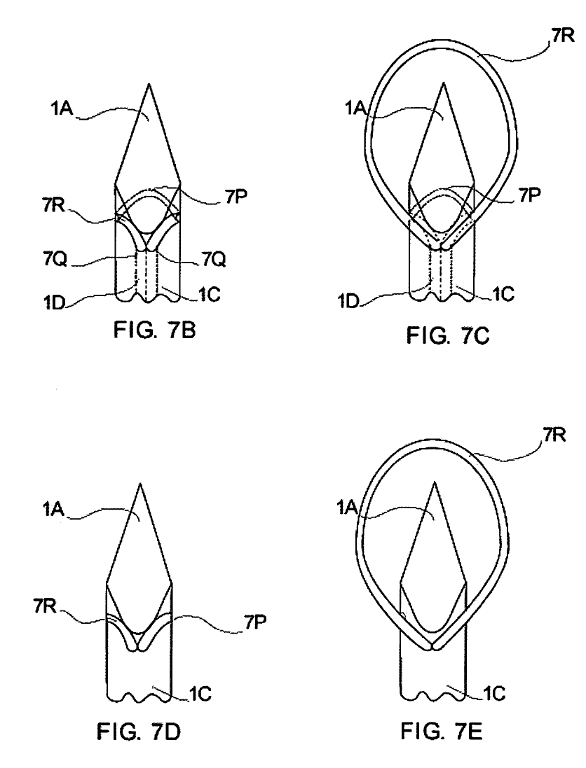

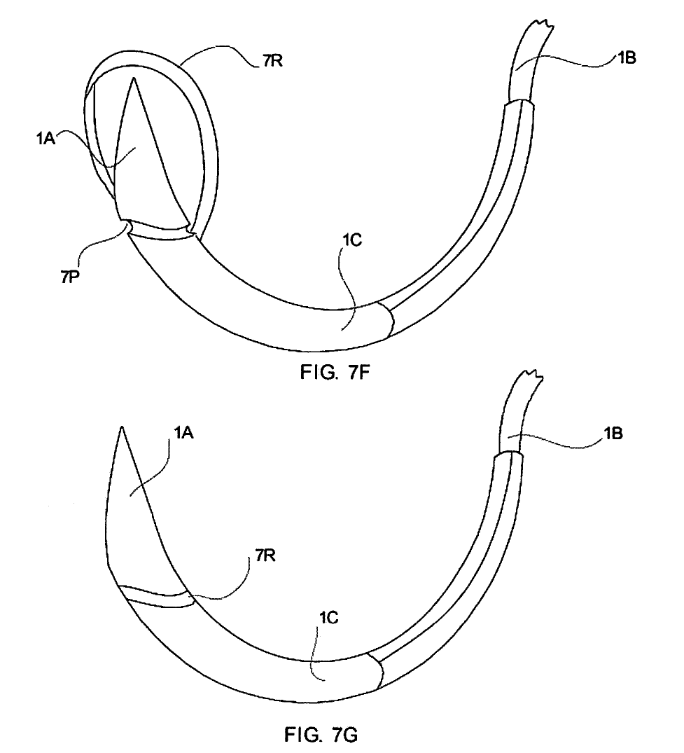

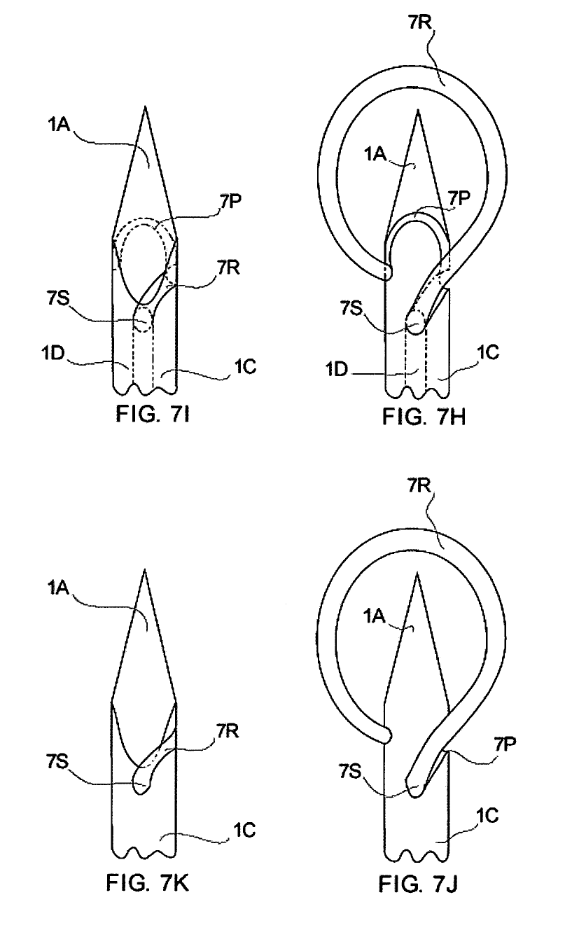

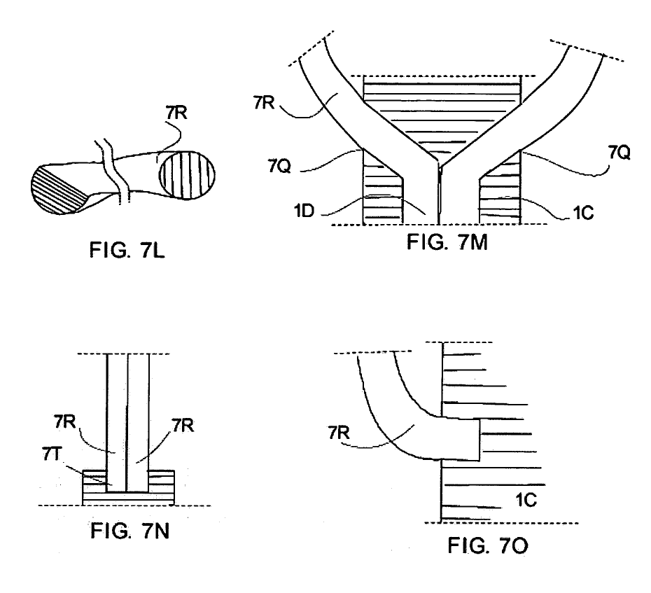

DS21; FIG. 7 and FIG. 7A illustrate a needle assembly with a wire loop sheath.

DS22; FIG. 7H-FIG. 7K illustrate various views and portions of a needle assembly with a wire loop sheath.

DS23; FIG. 7F-FIG. 7G illustrate various views and portions of a needle assembly with a wire loop sheath.

DS24; FIG. 7B-FIG. 7E illustrate various views and portions of a needle assembly with a wire loop sheath.

DS25; FIG. 7L-FIG. 7O illustrate various views and portions of a needle assembly with a wire loop sheath.

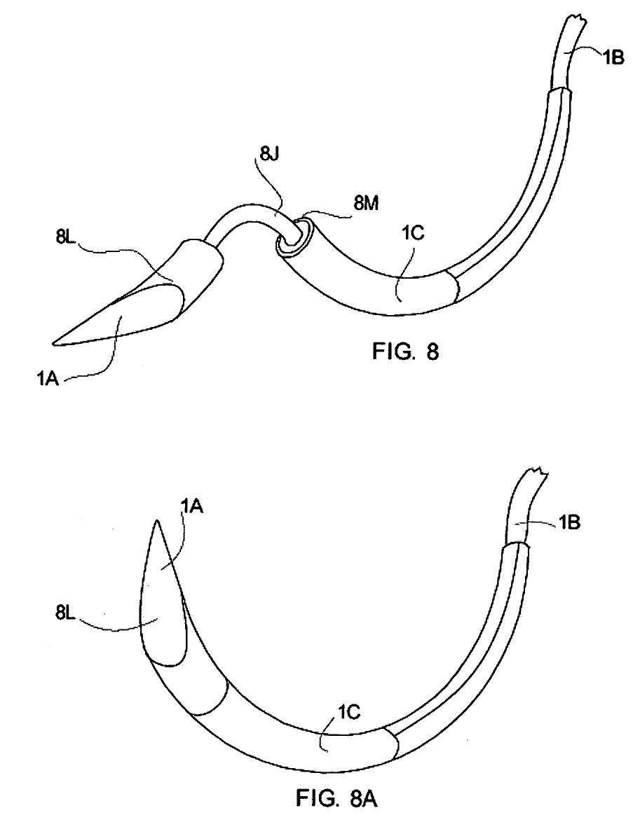

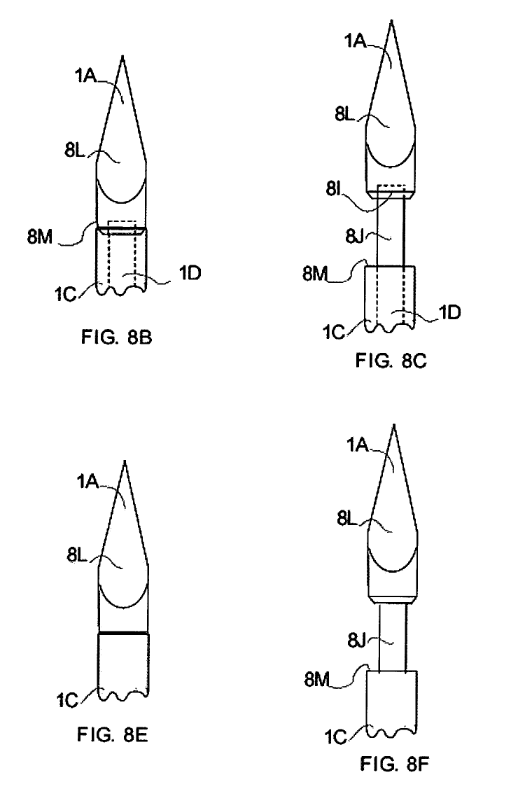

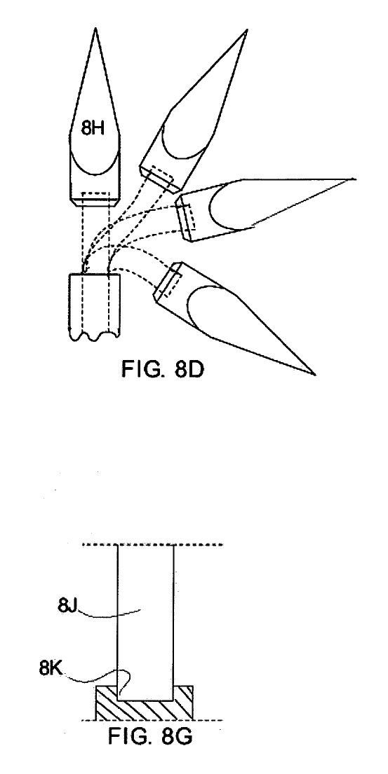

DS26; FIG. 8 and FIG. 8A illustrate a needle assembly with a break-away tip.

DS27; FIG. 8B, 8C, 8E, 8F illustrate various views and portions of a needle assembly with a break-away tip.

DS28; FIG. 8D, 8G illustrate various views and portions of a needle assembly with a break-away tip.

DS29; FIG. 2 illustrates a needle holder; FIG. 2A, and FIG. 2B illustrate opposite side views of a modified needle holder.

DS30; FIGS. 2.1, 2.a!, 2.3A, and 2.3B illustrate a modified needle holder.

DS31; FIGS. 2C, 2D, and 2E illustrate a modified needle holder.

DS32; FIGS. 2J, 2K, 2L, and 2M illustrate a modified needle holder tip.

DS33; FIG. 2F-FIG. 2H illustrate a modified needle holder with an initiation assembly and connection of segments of the needle holder body.

DS34; FIGS. 2.2, 2.2A, 2.2B, and 2.2C illustrate various initiators for initiating or activating an activation mechanism in a suture needle.

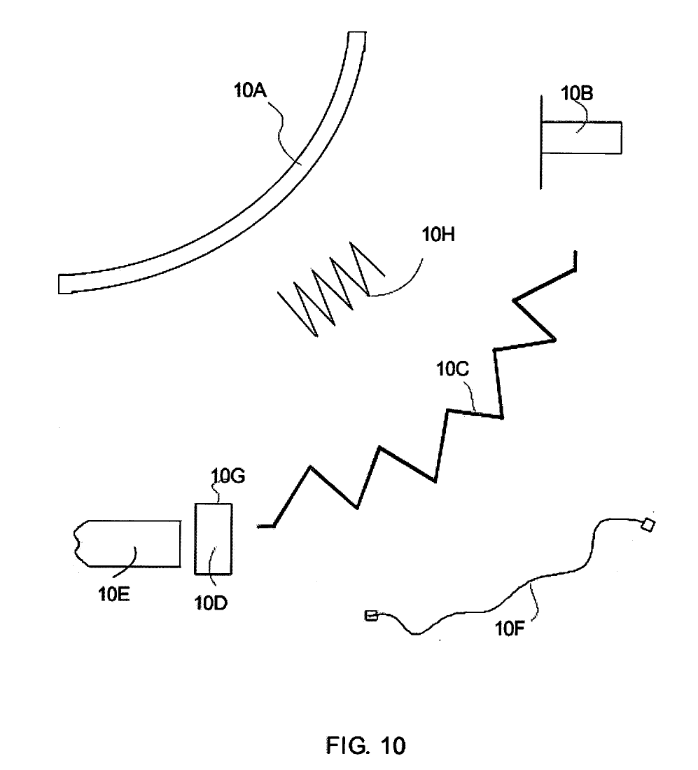

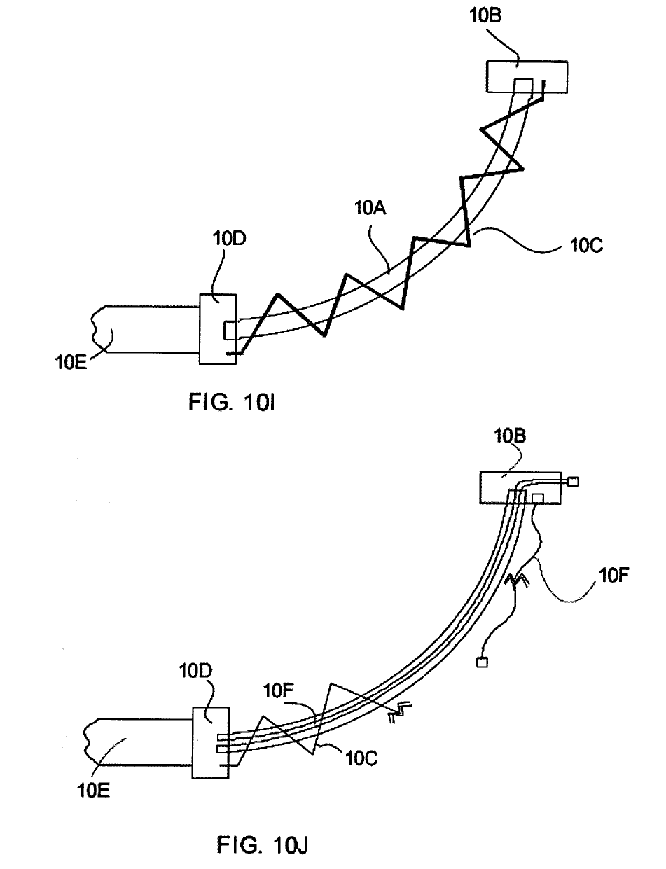

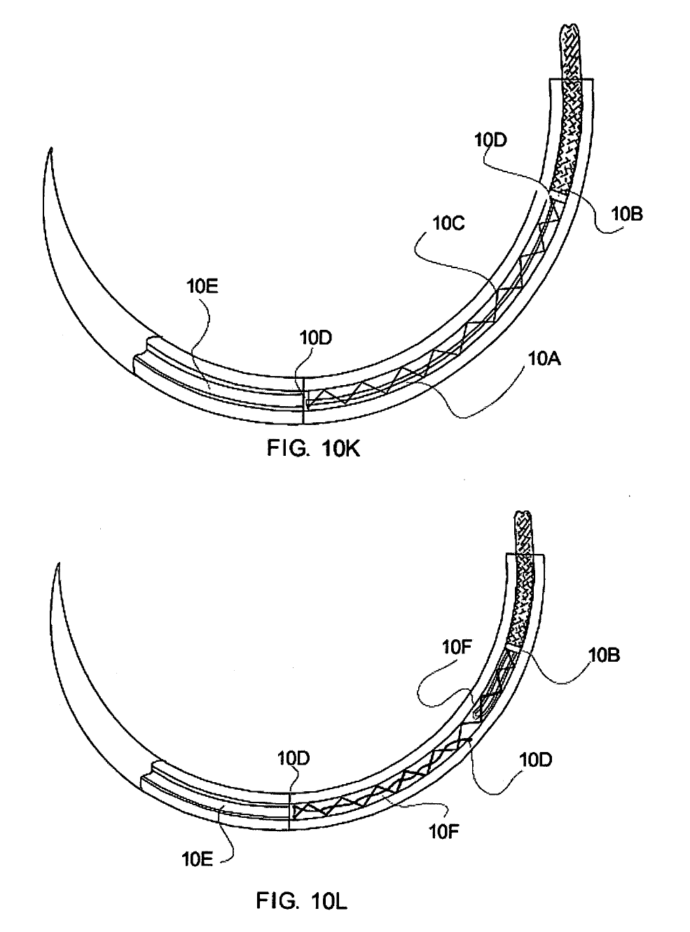

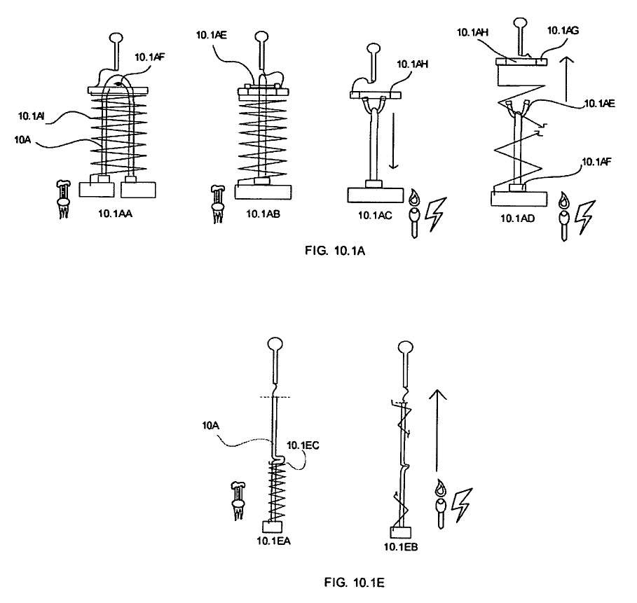

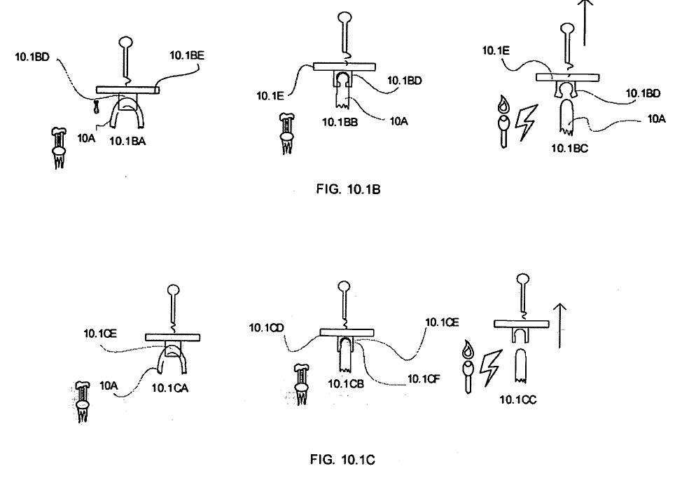

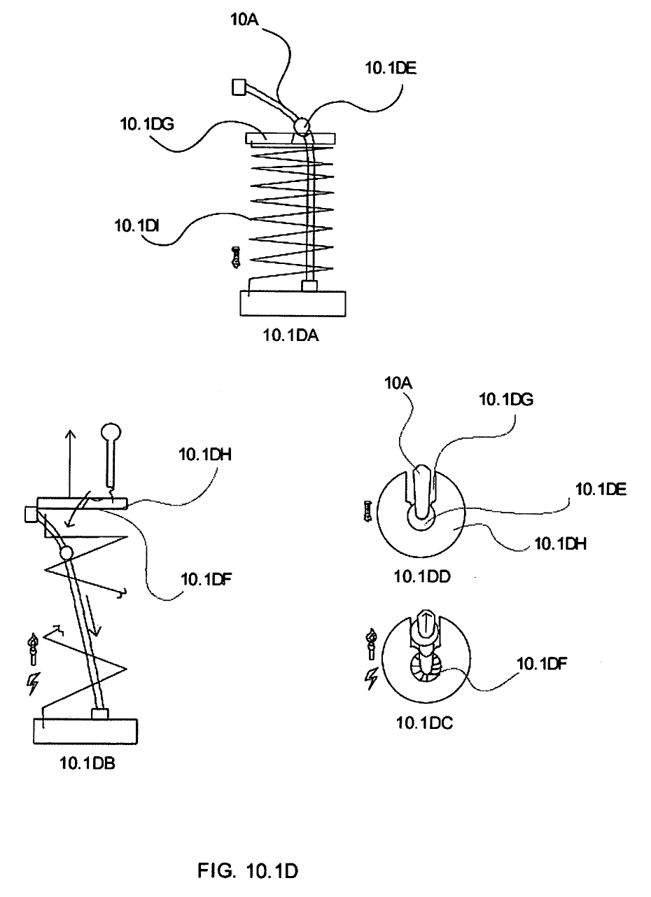

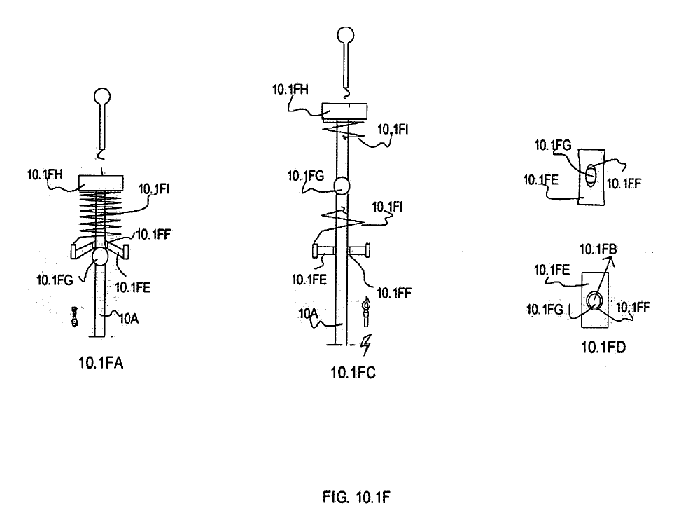

DS35; FIG. 10 illustrates components of an activator or mechanism of activation assembly for a suture needle with an activatable sheath

DS36; FIG. 10I, 10J illustrate an activation mechanism.

DS37; FIG. 10K, 10L illustrate activation mechanisms in needle bodies.

DS38; FIG. 10.1F illustrates variations of a sheath activator.

DS39; FIG. 10.1D illustrates variations of a sheath activator.

DS40; FIG. 10.1B and FIG. 10.1C illustrate variations of a sheath activator.

DS41; FIGS. 10.1A and !0.1E illustrate variations of a sheath activator.

DS42; FIG. 11 illustrates a needle body assembly variation.

DS43; FIG. 11B illustrates a needle body assembly variation.

DS44; FIG. 11D illustrates a needle body assembly variation.

DS45; FIG. 11A and FIG. 11AA illustrate views of a needle body assembly variation.

DS46; FIG. 11C and FIG. 11CA illustrate views of a needle body assembly variation.

DS47; FIG. 11E and FIG. 11EA illustrate views of a needle body assembly variation.

DS48; FIG. 11F and FIG. 11G illustrate one-piece and two-piece designs of a needle body assembly with top and bottom conduction plates.

DS49; FIGS. 11K, 11L, and 11M illustrate various attachments for a suture material near a needle body end.

DS50; FIGS. 11H, 11I, 11J, and 11T illustrate various approaches for joining needle body components.

Aspects and elements of the Figures are further set forth in Table 1-Table 11.

### DETAILED DESCRIPTION OF THE INVENTION

The invention may be further understood by the following non-limiting examples.

The disclosure herein, including the accompanying drawings, illustrates the variety of embodiments for needles, the activation mechanisms (e.g. an SMA assembly), and means for activating the activation mechanism. In an example, a needle holder serves not only to physically assist in holding the needle but also functions as a means for activating the activation mechanism. In an embodiment, a known needle holder can be modified with an attachment, or a presented novel needle holder can be used to activate the activation mechanism. In particular to take advantages of properties of SMA material, other modes of activation are included.

The drawings also illustrate various configurations of certain parts that may be useful in various SMA mechanisms. A particular configuration can be selected according to manufacturing possibilities, standardized regulations on durability, and consumer preference. Multiple possibilities and varieties of construction for an SMA mechanism of activation are also illustrated.

### EXAMPLE 1

### Safety Suture Needle Assembly

For the SMA mechanism of activation, two options are used. The choice of option can pertain to the method of construction of the device. If the needle is to have reversible on/off characteristics as described, the needle can incorporate a force exertion assembly. In the drawings, such a force exertion assembly is illustrated as a spring analogous to a conventional extension spring. A spring can be optionally constructed of any applicable and acceptable medical grade metal alloy. The spring is not necessarily confined to a metal spring configuration but can be constructed of a polymer material or configured by many other widely accepted means of placing a rebound tension on the activation mechanism itself.

If a needle of the invention does not incorporate on/off characteristics but namely has a one time or one way activation characteristic, a spring assembly may be optionally excluded. In a configuration where a spring is excluded, the activation mechanism relies solely on the properties of the SMA material to return to its preformed shape in order to exert the linear extension of the sheathing assembly to achieve a protective position such as over the needle body. An example of a situation where a spring is optionally included is as follows. A certain amount (e.g. a short or limited amount) of retraction may be desired to secure the tip firmly within a resting point/position, or a sheathing guard point/position within the sheath itself, as to entirely secure and encompass the sharp point of the needle. If this short amount of retraction is utilized to accomplish the desired effect, a small compression spring may be internally attached to the sheathing assembly or the mechanism of activation itself, in order to provide the retraction desired.

Elements of the needle body itself are represented as being of a suitable metal or alloy, optionally of acceptable standards. For certain device embodiments, elements of the needle body may need to be insulated from the electrical current which passes through it; high density polymers may be used to construct the outer needle body, or any of the other parts, depending on manufacturing tolerances and the design deemed as most desirable.

The general representation of the entire needle assembly can involve a hollow casing with certain parts resting inside the casing and certain other parts oriented around the inner parts so as to allow an electrical current to flow through efficiently, thereby facilitating a process of ohmic heating or so as to allow thermal heating by thermal conduction. Particular embodiments of the invention, however, are not limited to the only internal placement of certain parts. Particular variations for the needle assembly can allow construction and position of parts/elements at different points about the body of the needle assembly. Namely, it is possible to have the extension sheath externally located (e.g., as opposed to integrally flush with the needle body), and also it is possible to design the mechanism of activation as an externally attached unit separate from the main needle body itself, for example the SMA mechanism could run proximally to the suture material, distally from the tip of the needle.

The sheathing mechanisms shown support how to accomplish the sheathing/blunting effect desired; however, the mode of sheathing and/or blunting can also be achieved by other manifestations. For example, an externally placed SMA wire can run along the top side of the needle assembly; when treated such as by heat, the wire can return to a performed shape such as one that reaches a protective position at least partially covering the sharp point of the needle tip.

FIG. 1 illustrates a general needle body of a needle assembly of the invention. The shown needle shape is divided into three sections, the distal needle tip area in general represented by FIGS. 3-8, and each figure of the grouping 3-8 provides specific detail on a variety of possible sheathing/blunting assemblies. It is implied but not necessarily shown in certain diagrams that there can be a linear extension shaft that runs along a longitudinal axis within a cannula of the needle body, which connects the sheathing assembly to a mechanism of activation. In some representations of the present invention, the sheathing body is designed so as to be part of the extension shaft itself.

The shown needle shape in FIG. 1 is further divided into a middle section for clarity and is shown in greater detail in FIGS. 9-10 with mechanisms of activation. Another subdivision of the general needle is the proximal end with respect to an attachment point for the suture material and is detailed in FIG. 11. Generally these figures illustrate various ways to construct the external needle casing and attach the suture material to the needle body itself. One of ordinary skill in the art will appreciate that the parts can be constructed, joined, and manufactured by various techniques including commonly known practices.

The four figures grouped together as FIG. 1I, FIG. 1J, FIG. 1K, and FIG. 1L are presented occasionally throughout the drawings to clarify functional characteristics of the activator or mechanism of activation in relation to the needle assembly and whether or not activation of the activator is initiated by an initiator such as heat 1J, electricity 1K, or whether the activator is in a rest or default state with no heat or electrical energy being administered to it. The FIG. 1I refers generally to the presence of a blunting/sheath mechanism, and is shown occasionally throughout the drawings to reference the presence and or location of the blunting/sheathing assembly.

FIGS. 1E-1H illustrate usage of the needle assembly device. FIG. 1E represents the passing of the device between two persons and conveys the safe attribute of the blunted characteristic of the needle during transport (the tip variation of FIG. 8 is depicted solely for example). FIG. 1F shows the common practice of loading a needle assembly by an operator onto an art-known needle holder (further illustrated in FIG. 2.1) and shows the blunting characteristic of this specific example. FIG. 1G shows usage of the needle assembly in a common medical practice of tissue penetration or suturing with the sheathing/blunting blunting assembly (of the FIG. 8 variation) in an armed state. Here activation is achieved by an operator's left hand depressing a control button which is explained in the FIG. 2 set. FIG. 1H shows the release of the said control button and the result of deactivation or disarming which renders the needle assembly less likely to cause an accidental puncture. It is possible to begin the process of activation with either hand and from any angle (see FIG. 2.3A, FIG. 2.3B); for example, multiple switches can be incorporated.

The following describes some of the properties of the mechanism of activation and illustrates embodiments of the invention. See FIGS. 9A-9Q (in particular FIG. 9A, FIG. 9B, FIG. 9C, FIG. 9D, FIG. 9E (shown as encompassing aspects of FIG. 9A and FIG. 9C); FIG. 9F (shown as encompassing aspects of FIG. 9H, FIG. 9J, FIG. 9O); FIG. 9G (shown as encompassing aspects of FIG. 9L, FIG. 9N). The figures within FIG. 9A represent the characteristic of the SMA to be at rest with a preformed shape. FIG. 9A depicts an internal nitinol wire (not shown) inside a spring, where the spring is capable of exerting an opposing force to that of a force exhibited by the nitinol wire either when contracting or returning to an original position/shape. During a heat annealing process the alloy is raised to a temperature above its transition point, and then deformed at that point. Upon cooling, the alloy is then deformed again to the desired shape. When the alloy is heated at a later time, it has the special property to return to its previously set shape that was formed during the annealing process. FIG. 9O then shows the linear movement desired of the mechanism of activation with the application of heat or electricity to the material.

The vertical column within the brackets of 9G represents the length of the horizontal placed material. The vertical column of FIG. 9F represents a displacement distance that the sheath/blunting mechanism and/or extension assembly travels to reach a position at which the sheath/blunting mechanism is effectively protective with respect to the sharp aspect of a sharp object.

The vertical column of 9E represents a linear component of a spatial region where the sheathing/blunting mechanism is acting in a protective fashion. FIG. 9B illustrates a general needle shape in reference to its blunting/sheathing assembly, characteristics of the said assembly shown in FIG. 9A, and the location of the said blunting sheathing assembly relative to the sharp needle tip. Within FIG. 9B, FIG. 9P illustrates a configuration at rest; FIG. 9Q illustrates an activated configuration; the intended direction of travel of the sheathing/blunting mechanism during activation towards the activated state (here disarmed).

FIG. 9C illustrates the properties of the mechanism of activation with an extension spring for linear extension. FIG. 9H represents the needle assembly at rest with the SMA material at the inner core of an assembly of the SMA material and the spring. In a preferred configuration there are two connection points between the SMA component and the spring component of the assembly (such as at opposite ends of the SMA component).

FIG. 9L shows the contraction/retraction of the SMA material, which ranges from about 6%-10% of its overall length upon application of energy input to the SMA material. The SMA material contracts in length, and its diameter increases in a corresponding range. This fact is taken into consideration when constructing and inserting the activation mechanism assembly (and optionally a separate extension shaft, etc.) into the needle. FIG. 9J shows the extension of the SMA material by the force exerted upon it by the extension spring. The vertical column within the brackets of 9G represents the length of the horizontally displaced SMA material (or SMA and spring assembly). The vertical column of FIG. 9F represents the distance that the blunting mechanism and/or extension assembly has to travel to reach the point at which it effectively acts as a blunting/sheathing mechanism. The vertical column of 9E represents the area at which the sheathing/blunting mechanism is acting effectively with its purpose to at least partially shield the sharp object tip.

FIG. 9D illustrates a general needle shape and orientation of location with respect to its blunting/sheathing assembly, the SMA/spring assembly described in FIG. 9C, and how the blunting/sheathing assemblies location is affected in each state (rest, activation, returning to rest) and process (compression/extension of spring, contraction/expansion of SMA material, and relative displacement of length). FIG. 9K shows the sheath at rest, FIG. 9L shows the sheath being retracted during activation, and FIG. 9M shows the sheath being extended by the extension spring.

Suture Needle of FIG. 3

A general suture needle is illustrated in FIG. 3 and shows a radial/cylindraceous sheath assembly 3Q protruding distally from the needle, rendering the sharp point at least partially covered so as to protect the operator. FIG. 3A shows the same general needle with sheath 3Q at its retracted position. FIG. 3A-3I show specific defined views and the relation of the said parts to the assembly. The sheath has a vertical channel 3J which allows for the expansion 3P of the sheath in order to displace or slide over the needle tip upon retraction or extension (see FIG. 3I). Sheath 3Q comes to rest within the notch or space 3R which is formed from the main needle body. Channel 3J meets the elevated ridge 3O which is formed from the main needle body, in order to secure the sheath during its various sequences. The sheath 3Q is attached or is itself a unitary part of slider shaft 3K, which runs through an aperture 3N within the needle body itself 1C to the inner cannula 1D where slider shaft 3K is attached to a mechanism of activation (See various FIG. 10; FIG. 10, FIG. 10I, FIG. 10J, FIG. 10K, FIG. 10L). Sheath 3Q has a slightly rounded edge on the superior portion 3L so as to prevent the formation of an additional puncture hazard and also to allow for easy passage from notch 3R to its extended position. The edges of notch 3R including a tip proximal notch leading edge, a trailing edge, and/or edges near raised ridge 3O are made smooth (not shown) to facilitate the ease of transition of sheath 3Q to move freely from the extended to the retracted position. The sheath 3Q is stopped and makes a snug or tight junction upon meeting element 3M which is a resting ledge that is preferably substantially uniform. The sheath assembly disclosed can be adapted or modified to fit sharp object tips of a variety of tip sizes, shapes, and characteristics.

Suture Needle of FIG. 4

A general suture needle is illustrated in FIG. 4 and shows a sheath assembly 4V protruding distally from the needle, rendering the sharp point covered so as to protect the operator. FIG. 4A shows the same general needle with sheath 4V at its retracted position. FIG. 4B, FIG. 4C, FIG. 4D, and FIG. 4E illustrate specific defined views of the assembly and the relation of the parts to the assembly and needle tip 1A. The shield-like sheath 4V has a point or recess 4X on the bottom of the sheath. When the shield 4V moves over the needle tip (such as when used in the context of one time activation with a retraction spring 10H {See FIG. 10} within an assembly of the mechanism of action of FIG. 10), the needle tip comes to reside within a sharp tip catching point, element 4X, which “locks” the shield 4V on to the needle tip 1A. The shield 4V is blunted or slightly rounded with an arch or hood, element 4Y, about its outer perimeter, so as to not produce an unnecessarily hazardous sharp aspect in addition to the primarily hazardous tip.

Shield 4V is then connected to an extension 4W or is uniformly constructed as part of the extension itself. The extension 4W is then connected to the mechanism of activation; see various FIG. 10.

The shield 4V upon retraction comes to reside within a notch or aperture 4U of the main needle body. Upon retraction the shield and needle body 1C form a radially substantially uniform body, so that there is minimal opportunity for catching or unwanted drag from the needle during passage through tissue. In a preferred embodiment the entire needle is substantially flush or smooth with the shield in the retracted position. FIG. 4F shows a side view of the aperture 4U in relation to the needle body. The shielding mechanism and aperture are located close enough to the tip 1A so as to equate the amount of travel given from the extension and contraction of the assembly of activation and also so that the tip is not weakened by the aperture being placed to close to a narrow portion of the needle tip, which could possibly weaken the tip and needle body.

In order to guide the shield to its proper orientation upon retraction and or extension, an optional groove can be placed within or along the needle body and distally to the aperture that matches the extension shaft. When the activation or activation and extension mechanism is in movement, the groove would facilitate orientation of the shield into its proper alignment. FIGS. 4G, 4H, and 4I show in detail the shielding mechanism and extension shaft. The extension shaft 4W has one or more “catch points”, element 4Z, to reduce flexion of the shield if force is applied to the shield that may displace the shield to a spatial region away from a protecting position. The catch points 4Z are accepted by grooves 4AA disposed within the needle body so as to capture the sheath to facilitate the performance of the sheath in a secure manner. FIG. 4T gives a detailed view of resting points 4Z in relation to the catch point grooves 4AA. The shield can optionally be formed so that no sharp tip catch point exists, and the needle can be reversibly activated and deactivated multiple times by the retraction and extension of assemblies shown in FIGS. 4J-4S. The sheath assembly disclosed can be adapted or modified to fit sharp object tips of a variety of tip sizes, shapes, and characteristics.

Suture Needle of FIG. 5

A general round suture needle is presented in FIG. 5 (see also FIGS. 5A-5J), illustrating and shows a preformed wire ribbon or loop assembly 5N protruding distally from the top medial portion of the needle tip 1A through two exit apertures 5L and 5M. FIG. 5A shows the same needle assembly with the sheath/blunting mechanism at rest or retracted into the main needle body. If the needle is initially supplied in the state depicted in FIG. 5 with the sheath in a protective position with respect to the needle sharp end, this preset disarmed needle can then be activated to achieve an armed configuration with the sheath in a nonprotective position. FIG. 5B, FIG. 5C, FIG. 5D, and FIG. 5E show specific defined views of the assembly and the relation of parts to the assembly and needle tip 1A.

The sheath/blunting wire mechanism 5N can exit as an optionally sole unit from the main body extension shaft through a secondary aperture 5O, as two separate but joined wires to the main extension shaft. In this variation, the wire loop itself forms the extension shaft (note FIG. 7N, showing the two ends of the wire, connected to a slider plate, which is really the mechanism of activation). Alternatively, the exit mechanism can have a plate 5P covering the aperture that can have a single aperture or two apertures, 5L and 5M, depending on the formation of the preformed wire assembly 5N. There are many variations of exit mechanism configurations for this type of sheath/blunting mechanism in the retracted and activated positioning of the wires; a particular variation can be selected for purposes such as manufacturing efficiency or aesthetic considerations.

FIG. 5I shows a transparent lateral view of a secondary aperture 5O that is positioned at any angle to form an efficient and correct angle for exit of the sheath/blunting assembly. The blunting mechanism 5N is located within the shown area can retract when activated to an accepting groove 5K, and would rest at this location.

FIG. 5H demonstrates that the sheath/blunting assembly 5N can optionally have a characteristic of flexibility. The flexibility of 5N can be useful during the spatial translocation of moving over the needle tip point in order for a tight joining of the wire and the needle body itself 1A. The left panel of FIG. 5H shows a starting configuration of the sheath; in the middle panel, the inset arrows are shown as extending outwardly to reflect the flexible outward extension of segments of sheath 5N. The third or right panel has inset arrows pointing inwards relative to the sheath assembly to reflect the flexible inward extension of sheath 5N segments. Thus FIG. 5H demonstrates the potential lateral flexibility of the wire loop.

FIG. 5G illustrates an embodiment of the needle assembly with a portion of sheath wire 5N achieving substantial uniformity of surface continuity with a portion of needle body 1C due to sheath accepting channel 5K. In a preferred configuration, the accepting channel can receive substantially the entire sheath in a state that can optionally be referred to as a resting state or activated state depending on an initial preset condition of being armed or disarmed.

The sheath 5N can be constructed of suitable materials as known in the art. Note that the sheath wire need not necessarily be constructed only of nitinol or other SMA, nor does the sheath need to be constructed of the same material as extension shaft or activator components. The sheath wire can be plastic or nonplastic polymer. As in FIG. 5F, The sheath can have a full circle diameter shape such as a wire except optionally for a portion or segment that can rest within the accepting channel or groove which can be of a half circle shape. The sheath 5N can alternatively be of any shape necessary to create in conjunction with the needle body a substantially uniform exterior so as to reduce drag or snagging of the wire (FIG. 5G). For example, the sheath can be flattened like a ribbon, or composed of segments with different shapes.

Suture Needle of FIG. 6

A general round suture needle is presented in FIG. 6 with a blunted rod 6R having a blunt end surface 6S that projects from the tip 1A upon extension. The rod 6R can be a wire, bar, beam, or other object such as a blunting means. The exiting projection of the blunt rod 6R itself can be substantially along a hypothetically extended longitudinal axis of the sharp point tip 1A so as to provide a blunting body that a surface would come into contact with primarily as opposed to the sharp tip of the needle. The surface here for example can be a patient surface, medical worker human or clothing surface, needle operator human or clothing surface, or surgical environment surface. The blunt rod tip can protrude in any direction or from any segment of the needle body and not just from the needle tip end in the single direction along a longitudinal axis of the needle body.

FIG. 6A shows the blunted rod projection in a retracted position. In a preferred configuration, rod tip 6S is blunted just enough so as to not act as another sharp area but not be so blunted to allow excessive drag or snagging of a tissue subject to penetration. The blunted projection can be a singular unit that connects to the mechanism of activation assembly (FIG. 10) or can comprise or connect to a lateral extension 6T that connects to a general extension such as in FIG. 10. Specific views are detailed in FIG. 6B, FIG. 6C, FIG. 6D, FIG. 6E, FIG. 6G, and FIG. 6H regarding the needle assembly and details.

FIG. 6G illustrates a variation where the blunt rod has a curved or angled segment 6Q, so that upon extension/projection of rod 6R the projecting rod has a spring-like characteristic to facilitate positioning of the rod 6R and rod tip 6S towards a protective position over the needle tip. Upon retraction, a spring-like bending effect can be small enough so as to facilitate ease of retraction. In a configuration for one-way or reversible activation, the curved segment 6Q straightens sufficiently and fits snugly within the main lateral needle cannula 1D.

For a configuration with a one-time activation property, the blunted projection rod 6R can further comprise a resting catch point groove (or notch, aperture, channel, recession, or means for receiving) shown in FIGS. 6M and 6N. This catch point can serve to facilitate storage of the projecting blunt rod in a locked (e.g. at least partially secured) position and can be combined with the small retraction mechanism in order to fit the projection snugly on to the tip. In a variation, a small rebound tension mechanism is placed in the body of the needle in order to secure the needle tip into the “pocket.” A second aperture 6O can be formed as illustrated in FIG. 6F and positioned so as to allow an efficient exit angle for the projection rod.

FIGS. 6I-6L show specific views of the needle assembly with detail. Further embodiments of devices and methods can use an extending rod fixed at a point proximal to tip 1A. In such embodiments, the rod is free to move vertically upon extension and also distally to cover the needle tip and provide a blunting effect (see FIG. 6P). FIG. 6P is an illustration of the conception and can be modified or adapted as taught herein and/or would be understood by one of ordinary skill. In FIG. 6P, a particular embodiment illustrates that by application of force along the rod against a fixed point towards the needle tip, a loop or segment of the rod material is pushed with vertical displacement from a needle longitudinal axis and with horizontal displacement along such axis, wherein the horizontal displacement is beyond the needle tip. In a variation, the rod is nitinol and integrated with the activation mechanism (and integrated with distinct slider and extension components too, optionally).

Suture Needle of FIG. 7

A general round suture needle is presented in FIG. 7 with a projection sheath 7R which has a first end and a second end, where said first and second ends are fixed adjacently to a single point (in other words, proximal and equal to each other; see FIG. 7N). Alternatively, said first and second ends are fixed at a first lateral point and a second lateral point of the needle body, respectively (and distal to each other); see FIG. 7, and FIG. 7A-7J. The sheath can be a wire loop. In the first variation both ends of the wire loop projection are attached at or near the same point (FIG. 7N); where the point is disposed along either the main general extension shaft or can directly connect to the mechanism of activation at a defined point such as segment 7T. In a second variation, the two separated ends of the sheath extension can also be placed laterally, e.g. on directly opposite sides of the needle body, with separate exit holes/apertures 7Q. Apertures 7Q are optionally substantially unitary with the main lateral cannula 1D.

FIG. 7M shows a closer view with details. In the second variation the sheath projection 7R is fixed at the point of attachment to either a general main extension or the mechanism of activation at a sheath 7R proximal end and at a lateral fixation point located on the needle body itself (see FIG. 7O). The design allows for a sheath/wire to expand laterally and then distally; the sheath can have a preformed curved segment or shape that can extend in a manner as to provide the necessary blunting effect.

For both variations, the sheath/wire is of a shape so as to correspond to a tightly fitting resting point 7P (see FIG. 7, et alia) when retracted towards the main needle body. This is analogous to concepts described herein such as in various FIG. 5. FIG. 7L illustrates that a sheath, such as a wire (of metal, plastic, or polymer, etc.) is shaped in one or more dimensions such as diameter to correspond to the needle body and resting groove 7P. The placement of exit guide channel 7S can be selected based on one or more factors such as efficiency of angle allowing activation and/or retraction, and manufacturing methods. Similar to the concepts illustrated in various FIG. 5, a secondary guide channel with a plate covering and corresponding holes/apertures for the wire projection wire segments to exit can be implemented. FIGS. 7H to 7K illustrated detailed views of the assembly. In a variation, the sheath is nitinol and integrated with the activation mechanism (and integrated with distinct slider and extension components too, optionally).

Suture Needle of FIG. 8

A general round suture needle is presented in FIG. 8 with a break-away tip design where the tip breaks away from the main needle body upon activation or extension of the mechanism of activation. In a disarmed state, needle tip segment 8L is tethered or suspended from the needle body 1C by a flexible extension component 8J and is generally able to move freely except for the tethering constraint at a needle tip segment end. FIGS. 8A-8F illustrate the assembly with various views.

A distal tip 8M of the needle body proximal to a break point has a force dissemination contact surface or blunting means so as to not provide a sharp surface in addition to the needle sharp tip. Upon the retraction of the mechanism of activation, the needle tip can retract and be guided by a guide assembly. In one variation the guide assembly comprises a simple ridge and groove (not shown). In another variation there is an asymmetrically rigid (e.g. asymmetrically elastic) flexible extension member; upon application of force to the flexible extension member, the member is guided or directed to bend towards a particular side or angle to facilitate displacement of the needle tip towards the main needle body. In an example, the flexible extension member comprises two different materials of different stiffness.

When normal suturing function is desired as with a conventionally contiguous needle, for the present break-away needle the mechanism of activation can tightly secure the needle tip to the needle body so that a force exerted upon the needle during tissue penetration will not substantially disturb the ability of the needle tip to maintain a position of alignment and/or connection with respect to the needle body.

FIG. 8D illustrates the needle assembly with flexible extension and shows multiple device states with the break-away property. For example, the break-away needle tip hangs or leans towards one side of the needle body such as location 8H. FIG. 8G illustrates that flexible extension 8J can have a segment or end 8K that allows connection of flexible extension 8J with the main general extension. The connecting segment 8K and corresponding connections of the flexible extension to the needle body/main extension and to the needle point 8L can be constructed using techniques known in the art, and likewise for the connection between needle point 8L and the flexible member extension 8I.

It is noted that for clarification of terminology, the needle assembly can be activated to assume a disarmed state which might be considered an inactivated state; conversely, depending on preset conditions the needle can be activated to assume an armed state. Activation can optionally be reversible or one-way.

Additional Materials and Methods, Part I

FIG. 10 illustrates various materials and methods including parts for construction of embodiments of the present invention and variations. Construction is not necessarily limited to the disclosed parts specifically. The various FIG. 10 are intended to generally and in certain instances specifically provide information on embodiments, e.g. information on the mechanism of activation, structural properties and characteristics for certain functions, and materials.

FIG. 10 illustrates a shape memory alloy material as element 10A. In an embodiment the SMA can be nitinol or another alloy with similar properties, and various formulations thereof as disclosed herein and/or known to the art, and is preferably nitinol.

If the nitinol assembly is to achieve the desired contraction effect by ohmic heating, in an embodiment the assembly must have a positive and negative terminal end for the conduction of the electrical current and be electrically insulated in various aspects by traditional means. The transmission of the electrical current can be achieve through various means but is generally represented by the FIG. 10F such as with a conducting wire. A spring assembly itself, however, could act as a conductor. Other possibilities include that the nitinol could be filled with a conductive matrix; the wire could be routed through the nitinol (such as through a nitinol tube)l; or the distal end of a slider point attached to the nitinol could make contact with the positive side of the needle and the negative side. Moreover, any combination of the needle body and or the above parts or others can achieve the proper conduction of energy to the SMA material to allow function.

In an embodiment of a suture needle and needle holder combination, the contact points for electrical conduction ultimately are routed to the needle holder so that the property of ohmic heating can be exhibited. Therefore, a member/point 10B (FIG. 10) is placed in contact with an external contact point or surface of the needle body, and a corresponding member/point 10D is placed towards an opposite end of said SMA component 10A to complete a path.

For simplicity of design and in part due to size constraints, a preferred embodiment minimizes the necessary parts such as for electrical operation. The main body construction is formed so as to give separate conduction areas, namely positive and negative sides (optionally referred to as plates; these can be separate pieces of the needle body).

In an embodiment, a spring form 10C or variation thereof is constructed and retained so as to provide a sufficient exerting force (rebound tension) so as to extend the SMA material after contraction; the exerting force is also able to be overcome by the contraction of the SMA material.

In another embodiment, a spring 10H (FIG. 10) is used in the assembly to achieve a singular permanent (one time) activation for the sharp tip resting point mechanism. Upon activation of the needle assembly, the linear projection of a sheath/blunting mechanism is extended; a retraction spring contracts or compresses the mechanism so as to allow the resting point to arrive at its desired location, and provide a snug fit. A main extension shaft 10E is connected to a sheathing/blunting sub-assembly and connected to a distal contact point of the SMA sub-assembly or to a slider or translocation means that can freely move within a compartment of a needle body cannula 1C (the slider/translocation means can optionally act as one of the conduction points in relation to moving within the needle assembly and making contact with the main needle body conduction parts).

In embodiments, variations of the one time use feature can pertain to the property of nitinol to contract when heated. FIG. 10.1A (includes FIG. 10.1AA, FIG. 10.1AB, FIG. 10.1AC, and FIG. 10.1AD) illustrates an activator or activation mechanism. The activator comprises an SMA wire, sheet, or filament 10A, a crossbar 10.1AE of bendable material that is either part of or separate to an extendable retention plate 10.1AG which can act as a retaining device for an extension spring 10.1A. The crossbar assembly and retention plate form a unit that retain the spring from extending. The SMA wire is positioned such as in a loop over the crossbar. Upon contraction of the SMA wire, the SMA wire pulls the crossbar down or causes the crossbar to break free from at least one of one or more contact points 10.1AF with the slider 10.1AG, thereby pulling the crossbar 10.1AE down through an opening in the slider 10.1AH and releasing the tension of the spring 10.1AI which in turn is driven to extend linearly the extension plate 10.1BE which is operatively connected to the sheathing mechanism.

FIG. 10.1B and FIG. 10.1C illustrate similar mechanisms (comprised of parts illustrated in FIG. 10.1D and FIG. 10.1E) that can be under tension. In these embodiments, the SMA/nitinol component is attached to the slider plate by a crimp, or crimp fastening means (e.g. element 10.1BD in FIG. 101BA etc.) and thereby secured.

FIG. 10.1C (including FIG. 10.1CA, FIG. 10.1CB, and FIG. 10.1CC) illustrate other variations of an activator having an under-tension mechanism. In these embodiments

In these embodiments, the SMA/nitinol component is attached to the slider plate by an adhesive such as glue, a weld, or fastening means to extension plate 10.1CD, where a bond strength is sufficient to retain the spring yet fails or breaks upon contraction of the SMA component.

FIG. 10.1D (including FIG. 10.1DA, FIG. 10.1DB, FIG. 10.1DC, and FIG. 10.1DD) illustrate further variations of an activator having an under-tension mechanism. In these embodiments, the SMA/nitinol mechanism involves a slider plate with a groove 10.1DG. The SMA component 10A can extend through slider plate groove 10.1DG, and the SMA 10A has an attached retention point 10.1DE permitting SMA 10A to rest firmly in slot 10.DF. When the SMA 10A contracts, it detaches or “pops” the retention point from the resting spot and slips the retention point 10.1DE through the corresponding groove 10.1DG within the slider plate, thereby releasing the plate which is under tension form the spring 10.1DI which extends the slider plate. The slider plate is ultimately connected to the extension or sheathing/blunting assembly.

FIG. 10.1E (including FIG. 10.1EA and FIG. 10.1EB) illustrate a further variation. In this variation, a bend in the SMA component 10A acts as a retaining area of the activation mechanism which is holding an extension spring under tension. Upon contraction, the SMA component releases the spring, thereby linearly extending or projecting the sheathing mechanism.

FIG. 10.1F (including FIG. 10.1FA, FIG. 10.1FB, and FIG. 10.1FC) illustrate other variations of an activator under tension. In these variations there is an SMA plate 10.1FE that is preformed in a bent configuration with an aperture/hole that is deformed 10.1FF when the activator is in a bended state so as to retain an extension shaft with a retaining point 10.1FG. The retaining point 10.1FG is unable to slide through the aperture/hole 10.1FF until the SMA plate 10.1FE is contracted. Contraction allows the opening up of the joint 10.1FF which allows the retaining point 10.1FG to slide through, thereby releasing the tension bound spring which is connected to the linear extension assembly.

In an embodiment, there can be a relatively simple device and design allowing elimination of many small and intricately engineered parts. The device can incorporate the function of one time activation, pertaining to the linear extension of a deformed nitinol wire that is activated by an external means. As disclosed herein, however, many activation mechanisms can be applied. The present inventor believes that the most efficient manifestation may require a lone deformed SMA shaped component which would achieve a net extension in a linear dimension and thereby extend or project a sheathing mechanism or be unitary in serving as an activating/extending and sheathing mechanism.

FIG. 10I, FIG. 10J, FIG. 10K, and FIG. 10L illustrate the general orientations of the assemblies of activation in relation to the external needle body parts and certain attachments, connections, or fixations of the elements without necessarily being limiting to certain areas or sequences of placement of parts or general assembly in manufacturing.

Additional Materials and Methods, Part II

The various figures in the set of FIG. 11 (e.g. FIG. 11, FIG. 11A, FIG. 11AA, FIG. 11B, FIG. 11C, FIG. 11CA, FIG. 11D, FIG. 11E, FIG. 11EA, FIG. 11G, FIG. 11F, FIG. 11H, FIG. 11I, FIG. 11J, FIG. 11K, FIG. 11L, FIG. 11M) illustrate the various assembly and construction of the needle body for purposes relating to embodiments of the invention, for example, for the needle body to conduct electricity, be structurally sound, and be able to retain device components or mechanisms (some of which are internally disposed in said needle body assembly) such as an activator assembly, extension shaft assembly, and sheath assembly. It is recognized that the construction and manufacturing relating to devices and methods of the present invention can accommodate adaptations and modifications as disclosed herein and as would be understood from the disclosure herein by techniques and knowledge available to one of ordinary skill in the art.

FIG. 11 illustrates a general suture needle in a conventionally curved form. The exterior main needle body can be manufactured according to techniques as known in the art. In an embodiment the main needle body and its accessories are formed separately form a top conduction plate 11N and a bottom conduction plate 11O, wherein the top and bottom conduction plates are affixed to a top needle body outer surface and bottom needle body outer surface respectively. The needle body is further formed so as to retain two proximal side portions 11P and 11Q that can each run laterally about half the length of the needle. In variations, the conductive aspect such as the side portions can extend along the needle body from about 5% to about 95% of the length of the needle body so as to allow a variety of locations for grasping by a holder or for transferring energy. The needle body can have a needle body core section removed, leaving two side walls 11P and 11Q in order to make space available for the conduction plates and provide attachment surfaces or areas for joining of the conduction plates to the needle body. The side panels and the conduction plates are joined using known means such as adhesive, crimping, welding, devices and methods shown in FIG. 11M or FIG. 11H, or means of fastening (alternatively, a needle body instrument is initially molded or forged to have desired capabilities) and are insulated as understood in the art, for example with current technology such as Teflon or plastic coating or standard means.

The preceding parts together form an exterior needle body as a whole thereby generating an internal compartment or hollow cannula 1D. On a conduction plate there can be a conducting surface to allow conduction of electricity and contact with a means for supplying electricity or to serve as an attachment abutment for connection to a suture material 11R. These separate parts are made and connected so as to provide a proper conduction pathway to transmit the necessary electrical current or so as to act as a retainer for an internal sub-assembly such as an activation mechanism.

FIG. 11A, and FIG. 11AA illustrate views of assembly of the needle body. FIG. 11B illustrates a main needle body design having only a top conduction plate to serve as an electrical terminal end as the main needle body can act itself like a conduction plate itself as a second terminal end of a circuit. FIGS. 11C and 11CA describe views of such an assembly.

FIG. 11D illustrates a main needle body comprising three separate pieces; a top plate 11N, a bottom plate 11O, and the main needle distal needle body 1C. The pieces are connected to form a whole such as by connecting the top and bottom plate with fastening means to form a top and bottom plate combination and connecting the combination to the body 1C using the same or different fastening means. Optionally the needle body 1C can be configured to allow partial overlap such as by comprising a half-cylinder segment receptive for either a top plate or bottom plate. FIGS. 11E and 11EA illustrate and describe views of this concept.

FIG. 11G illustrates a simple manifestation of a needle body using construction of only one piece. Such a needle body can be sufficient to accept some sub-assemblies such as certain mechanisms of activation. This design can be suitable for certain one time use activation mechanism involving stylizing the shape characteristic of an SMA component.

FIG. 11F shows a two piece design of a top plate that runs along the length of the needle body and a bottom plate which fully corresponds to the top plate. The joining of various parts can be accomplished for example with various means, including adhesives, crimps, welds, and also the utilization of new means according to this context. FIG. 11L gives a close up, of ridges, teeth, or frictional grasping means 11V that can be disposed along the an interior side of each of the top and bottom plates in order to give a grasping surface to retain a suture material. FIG. 11I shows a close up cutaway view of this potion of the needle in relation to the bottom and top plates, the conduction and attachment point 11R, and other pieces.

FIG. 11H illustrates utilization of an annular or partial ring-like fixture to secure or encompass the relevant parts of the needle assembly. Such a fixture can retain all of the pieces as one, for example by crimping the annular fixture 11T or by other fastening means, such as gluing, welding, etc. The corresponding portions 11U of the needle body parts are optionally manufactured to accompany the rings so as to minimize the providing of a surface allowing snagging, drag, or other unwanted effect. FIG. 11J illustrates a needle assembly end that has suture material in place with the described parts.

FIG. 11M illustrates channeling and notching of the corresponding portions of the needle body as a way to connect/assemble the parts. FIG. 11K illustrates crimping of a proximal end of the needle that is typically used for retention of suture material.

Regarding certain needle body parts, e.g. the needle main body, conducting plates, annular fixture, etc. a variety of conventional materials can be used as known in the art such as steel, stainless steel, and/or polycarbonate among others.

### EXAMPLE 2

### Safety Suture Apparatus Comprising a Needle and a Needle Holder

Needles of the invention are used in conjunction with a needle holder.

FIG. 2 (PRIOR ART) shows a front view of a general widely used needle holder of any size. FIGS. 2A and 2B (not prior art) illustrate opposite sides of a modified needle holder with a holder initiation assembly for attachment that carries out the function of initiating activation of the suture needle assembly by the application of heat or electricity.

The holder initiation assembly for attachment to an existing needle holder is further illustrated in FIG. 2C, FIG. 2D, and FIG. 2E. The holder initiation assembly has a vertical top portion 2P and a vertically corresponding bottom portion 2Q. The 2P and 2Q portions come together in an aligned fashion (see FIG. 2F, FIG. 2G, and FIG. 2H). This holder initiation assembly mechanism is designed so as to fit a variety of sizes of needle holders by utilizing attachment securing points 2Y (See FIG. 2C, FIG. 2E) which can fit over the corresponding portion of a needle holder. The holder initiation assembly portions 2P and 2Q can join together with an adjustably tightening ratcheting mechanism 2X comprising an upper portion 2Z (connected to 2P) that is notched for accepting the corresponding screw mechanism 2AA that is fixated within 2Q. The assembly ratchets together as illustrated by sequential portrayal of FIG. 2F, FIG. 2G, and FIG. 2H, by the tightening of the fixed assembly 2AA which has a shaft that is adjustable by the insertion of a standard or Alan wrench. Other adjustable and nonadjustable connecting means can be used for adjoining 2P with 2Q such as clip-locks and other fastening means.

The tip segment 2R of the 2P portion can be hollow in nature so as to allow the insertion of the corresponding needle holder tip sequence (FIG. 2J, FIG. 2K, FIG. 2L) shown in FIG. 2I. The holder initiation assembly locks on to a conventionally existing needle holder by tightening the ratcheting mechanism and is adaptable to different sized needle holders. The secondary tip appendage 2R can also be hollow in nature and fit over the second needle tip holder portion or “jaw” sequence shown in FIG. 2I (including FIG. 2J, FIG. 2K, and FIG. 2L showing an ordered sequence of events). This tip appendage is retained by a screw mechanism 2M (element 2M as opposed to FIG. 2M) that can be secured onto the second needle holder tip 2BB or screw directly into a receiving cavity or hole 2O that is disposed within the needle holder tip 2BB. The secondary tip of the said assembly is connected to the main upper body of the assembly 2P by a small wire 2U or means for allowing insulated transmission of electrical current to the conductive surface 2V.

As an overview explanation, the holder initiation assembly attachment has two attachment contact tips. A first attachment contact tip is element 2R, and a second attachment contact tip is a tip portion of element 2P. These two attachment contact tips fit over the needle holder tips 2W and 2BB (or insertionally receive 2W and 2BB) and provide conductive surface 2V for the transmission of electricity to the needle body or have internally located heating elements 2.2 that provide heat for initiation of the needle body mechanism of activation.

The needle holder initiation attachment can have an internal power supply 2U and an optionally simple internal circuit and circuitry (not shown). Both the power supply and the circuit (not shown) can be located apart from the needle holder unit (See FIG. 2.2C) and be located at a fixed point in a different power supply unit 2.2D with controls 2.2E, a cord 2.2G that connects to the needle holder, and an attachment plug 2.2F, that corresponds to the cord. The circuit is established as understood in the art by optionally a simple circuit of the pulse width modulation variety or other variety so as to control the nominal amount of voltage or current required. The circuit and controls are preferably adapted to prevent the overheating or malfunction of the assembly of activation. The circuit can have a regulatory mechanism, for example a control switch or button 2S, or means for circuit regulation that can optionally be ergonomically located to allow easy regulation by the operator in order to initiate the mechanism of activation ultimately residing in the needle.

In a particular example, the needle holder initiation attachment is of unitary construction and fulfills all necessary characteristics by incorporating the needed assemblies into one needle holder mechanism (See FIG. 2.1 and FIG. 2.1A).

Further devices and methods for initiation of activation include but are not limited to the following: a container of warm water, saline, or other fluid (FIG. 2.2B), a hot plate (FIG. 2.2A) with an internal or external power supply (not shown), circuit (not shown), and heating element (not shown).

In FIG. 2.3A and FIG. 2.4B, a needle holder variation is shown. In this variation, an operator can normally insert a thumb and finger (such as a forefinger) through the holes of the needle holder. At or near a point where the opposing fingers (such as thumb and finger) tend to naturally come together, a switch is placed. This switch can optionally have two segments or halves and be activated by a clamping of the needle holder.

Various combinations of configurations of the above assemblies used in conjunction with the mechanism of action of the needle itself can be used.

### EXAMPLE 3

### Safety Suture Needle With Unifying Element

Disclosed is a needle featuring a unifying element. The unifying element can provide reinforcement in the presence of adversely stressful forces that may otherwise result in cracking or dissociation of a part of the needle from another part or the remainder of the needle body. In a needle of the invention, an SMA component can be integrated to function as a relatively pliable shaft that is optionally placed centrally but nonetheless disposed along a portion or the entire length of a longitudinal axis of the needle. In case of accidental breakage, the shaft holds at least one or more broken pieces of the needle body together so as to not allow the broken pieces to fall, lodge, or be lost within a body tissue.

### EXAMPLE 4

### Safety Suture Kits

Kit forms of the invention are provided. A safety suture kit comprises a needle of the invention and suture material. Another kit comprises a needle and needle holder of the invention and suture material. Another kit comprises a needle, modified needle holder, and suture material.

TABLE 1
Information on FIG. 1 set.

| | Information |
|---|---|
| FIG. | |
| 1E- | Figure representing the passing of needle between two persons. |
| 1F- | Figure representing the loading of needle onto a needle holder. |
| 1G- | Penetration of needle through tissue. |
| 1H- | Disarming of needle. |
| 1I- | Indicates location of general blunting mechanism during stages |
| | of activation sequence. |
| 1J- | Represents the inactive state of mechanism. |
| 1K- | Application of electrical current for activation of mechanism. |
| 1L- | Application of heat for activation of mechanism. |
| Element | |
| 1A- | Needle Tip (sharp point). |
| 1B- | Suture Material. |
| 1C- | Needle Body. |
| 1D- | Main Lateral Cannula (Hollowed out bore). |

TABLE 2
Information on FIG. 2 set.

| | Information |
|---|---|
| FIG. | |
| 2- | Existing needle holder. |
| 2A- | Right side view of assembly to needle holder. |
| 2B- | Left side view of assembly to needle holder. |
| 2C- | Right side view of assembly. |
| 2D- | Left side view of assembly. |
| 2E- | Top view of assembly, with secondary attachment |
| | appendage shown in top view. |
| 2F- | Superior and inferior sections aligned for assembly. |
| 2G- | Insertion of the two sections. |
| 2H- | Joining of the two sections. |
| 2I- | Attachment sequence of the needle holder tip and secondary tip |
| | attachment. |
| 2J- | Aligned sections. |
| 2K- | Secondary tip appendage aligned with needle holder tip, |
| | beginning of attachment. |
| 2L- | Secondary tip appendage secured. |
| 2M- | Secondary tip attachment mechanism. |
| 2.1- | Lateral view of needle holder. |
| 2.1A- | Left side view of needle holder. |
| 2.2- | Cutaway view of needle holder with heating element in the tip |
| | for means of activation (heating element not shown). |
| 2.2B- | View of a nonspecific container of hot water. |
| 2.2A- | View of a nonspecific hot plate with either internal or external |
| | power supply, and Internal heating element. |
| 2.2C- | Needle holder with power supply separate from holder unit |
| 2.3A- | Side View specialized needle holder showing potential switch |
| | placement and physical properties. |
| 2.3B- | Front View specialized needle holder showing potential |
| | switch placement and physical properties. |
| Element | |
| 2N- | Secondary tip attachment bolt. |
| 2O- | Modified secondary tip predrilled hole. |
| 2P- | Upper section of assembly. |
| 2Q- | Lower section of assembly. |
| 2R- | Secondary tip appendage. |
| 2S- | Activation switch. |
| 2T- | Wire connector to secondary tip from main assembly body. |
| 2U- | Battery power supply. |
| 2V- | Conductive surface. |
| 2W- | Primary needle holder tip. |
| 2X- | Ridge showing the elevated tensioning mechanism area. |
| 2Y- | Ridge and groove for assembly retention on needle holder. |
| 2Z- | Top horizontally grooved rail for tensioning mechanism. |
| 2AA- | Fixated tensioning assembly. |
| 2BB- | Needle holder secondary tip. |
| 2.2D- | External power supply unit. |
| 2.2E- | Power supply controls. |
| 2.2F- | Plug connector for power supply and needle holder. |
| 2.2G- | Power cord. |
| 2.4- | Switch located in finger slots |
| 2.4A- | Protruding activator switch body |
| 2.4B- | Thumb depression switch |
| 2.4C- | Vertical switch. |

TABLE 3
Information on FIG. 3 set.

| | Information |
|---|---|
| FIG. | |
| 3- | Solid left perspective view, of general suture needle and inactive |
| | protective sheathing body tip variation. |
| 3A- | Solid left perspective view, of general suture needle and |
| | activated protective sheathing body. |
| 3B- | Solid Front orthogonal view of active sheathing body and |
| | needle tip. |
| 3C- | Transparent front orthogonal cutaway view of inactive sheathing |
| | body and needle tip. |
| 3D- | Solid rear orthogonal view of activated sheathing body and |
| | needle tip. |
| 3E- | Solid front orthogonal view of inactivated sheathing body and |
| | needle tip. |
| 3F- | Cross sectional view of needle holder tip point appendage. |
| 3G- | Solid front view of activated needle tip, showing resting sheath |
| | in relation to guide channel and guide ridge. |
| 3H- | Exploded isometric view of actual sheath. |
| 3I- | Representing 3 segments of sheath retraction, and extension in |
| | the reverse process |
| Element | |
| 3J- | Vertical expansion groove within sheath. |
| 3K- | Sheath assembly extension shaft. |
| 3L- | Figure showing the slightly blunted and rounded edges of the |
| | sheath body. |
| 3M- | Resting ledge of main needle body, for resting sheath body, |
| | upon retraction. |
| 3N- | Vertical opening from main needle body for raised attachment |
| | of main stem. |
| 3O- | Lateral ridge guide, which accompanies the vertical channel |
| | located within the sheath body. |
| 3P- | Expanding sheath body, sliding over the needle tip. |
| 3Q- | Sheath assembly. |
| 3R- | Sheath retraction resting area. |

TABLE 4
Information on FIG. 4 set.

| | Information |
|---|---|
| FIG. | |
| 4- | Solid Left perspective view of general suture needle and inactivated shielding |
| | assembly. |
| 4A- | Solid Left perspective view of general suture needle and activated shielding assembly. |
| 4B- | Transparent front orthogonal view of general suture needle tip and inactivated |
| | shielding mechanism. |
| 4C- | Transparent front orthogonal view of general suture needle tip and activated shielding |
| | mechanism. |
| 4D- | Solid front orthogonal view of general suture needle tip and activated shielding |
| | mechanism. |
| 4E- | Solid front orthogonal view of general suture needle tip and inactivated shielding |
| | mechanism. |
| 4F- | Cutaway transparent side view of general suture needle tip body, and sheath resting |
| | point aperture. |
| 4G- | Solid top view of shielding assembly and cutaway extension shaft. |
| 4H- | Solid bottom view of shielding assembly and extension shaft. |
| 4I- | Left perspective view of shielding assembly. |
| 4J- | Solid Left perspective view of general suture needle and inactivated shielding |
| | assembly, without needle tip guard catch point. |
| 4K- | Solid Left perspective view of general suture needle and activated shielding assembly |
| | without needle tip guard catch point. |
| 4L- | Transparent front orthogonal view of general suture needle tip and activated shielding |
| | mechanism without needle tip guard catch point. |
| 4M- | Transparent front orthogonal view of general suture needle tip and activated shielding |
| | mechanism without needle tip guard catch point. |
| 4N- | Solid front orthogonal view of general suture needle tip and activated shielding |
| | mechanism without needle tip guard catch point. |
| 4O- | Solid front orthogonal view of general suture needle tip and inactivated shielding |
| | mechanism without needle tip guard catch point. |
| 4P- | Cutaway side view of general suture needle tip body, and sheath resting point |
| | aperture without needle tip guard catch point. |
| 4Q- | Solid top view of shielding assembly and cutaway extension shaft without needle tip |
| | guard catch point. |
| 4R- | Solid bottom view of shielding assembly and extension shaft without needle tip guard |
| | catch point. |
| 4S- | Left perspective view of shielding assembly without needle tip catch point. |
| 4T- | Close-up left cutaway side view of resting point groove and extensions in relation to |
| | the needle body, and shown intended mechanism of action, and shield retention. |
| Element | |
| 4U- | Sheath resting point aperture on needle body. |
| 4V- | Sheath assembly in general. |
| 4W- | Sheath assembly extension shaft. |
| 4X- | Sharp needle tip guard catch point. |
| 4Y- | Slightly blunted edge of sheath. |
| 4Z- | Sheath resting point extensions. |
| 4AA- | Sheath resting point grooves. |

TABLE 5
Information on FIG. 5 set.

| | Information |
|---|---|
| FIG. | |
| 5- | Solid left perspective view, of general suture needle and |
| | inactivated protective sheathing loop tip variation. |
| 5A- | Solid left perspective view, of general suture needle and |
| | activated protective sheathing loop tip variation. |
| 5B- | Transparent front orthogonal view of cutaway activated loop |
| | variation. |
| 5C- | Transparent front orthogonal view of cutaway inactivated |
| | loop variation. |
| 5D- | Solid front orthogonal view of cutaway activated loop variation. |
| 5E- | Solid front orthogonal view of cutaway inactivated loop |
| | variation. |
| 5F- | Close up view of ½ circle wire variation drawn to normal round |
| | extrusion shape. |
| 5G- | Showing the resting of the protective wire body, within the |
| | nesting groove. |
| 5H- | Showing the flexible properties of sheathing assembly, for |
| | accommodating extension and contraction of sheathing |
| | mechanism, so that the protective loop fits uniformly |
| | within the resting groove. |
| 5I- | Transparent lateral view of secondary guide channel variation. |
| Element | |
| 5J- | Secondar uide channel coverin plate with secondary holes. |
| 5K- | Circumferential Nesting groove for accepting loop when |
| | activated. |
| 5L- | Left wire loop exit aperture for protective body extension. |
| 5M- | Right wire loop exit aperture for protective body extension. |
| 5N- | Preformed section of protective loop sheathing body. |
| 50- | Secondary guide channel area. |
| 5P- | Aperture cover plate. |

TABLE 6
Information on FIG. 6 set.

| | Information |
|---|---|
| FIG. | |
| 6- | Solid left perspective view, of general suture needle and inactivated protective blunted |
| | wire projection tip variation. |
| 6A- | Solid left perspective view, of general suture needle and activated protective blunted |
| | wire projection tip variation. |
| 6B- | Transparent front orthogonal view of activated blunted wire projection tip. |
| 6C- | Transparent front orthogonal view of inactivated blunted wire projection tip. |
| 6D- | Solid front orthogonal view of activated blunted wire projection tip. |
| 6E- | Solid front orthogonal view of inactivated blunted wire projection tip. |
| 6F- | Left cross sectional side orthogonal view of secondary guide channel variation. |
| 6G- | Solid left perspective view, of general suture needle and inactivated protective blunted |
| | wire projection tip, with preformed curve, and sharp point nesting area variation. |
| 6H- | Solid left perspective view, of general suture needle and activated protective blunted |
| | wire projection tip, with preformed curve, and sharp point nesting area variation. |
| 6I- | Transparent front orthogonal view of activated blunted wire projection tip. |
| 6J- | Transparent front orthogonal view of inactivated blunted wire projection tip, showing |
| | the sharp point nesting area. |
| 6K- | Solid front orthogonal view of activated blunted wire projection tip. |
| 6L- | Solid front orthogonal view of inactivated blunted wire projection tip. |
| 6M- | Transparent lateral orthogonal view of inactivated wire tip, and sharp point nesting |
| | area. |
| 6N- | Orthogonal close-up view of sharp tip nesting area. |
| 6P- | Solid left perspective view of the SMA inlaid into a general suture needle, can also |
| | represent a needle with a fixed anchor point sheathing wire. |
| 6U- | Rear Left perspective view of general needle, with in laid Nitinol wire. |
| Element | |
| 6O- | Secondary guide channel. |
| 6Q- | Blunted extension preformed curve. |
| 6R- | Blunted extension of main lateral extension wire. |
| 6S- | Blunted tip portion. |
| 6T- | Attached extension wire. |

TABLE 7
Information on FIG. 7 set.

| | Information |
|---|---|
| FIG. | |
| 7 | Solid left perspective view, of general suture needle and inactivated protective non- |
| | preformed wire loop projection variation. |
| 7A- | Solid left perspective view, of general suture needle and activated protective non- |
| | preformed wire loop projection variation. |
| 7B- | Transparent front orthogonal view of activated wire loop projection. |
| 7C- | Transparent front orthogonal view of inactivated wire loop projection. |
| 7D- | Solid front orthogonal view of activated wire loop projection. |
| 7E- | Solid front orthogonal view of activated wire loop projection. |
| 7F- | Solid left perspective view, of general suture needle and inactivated protective non- |
| | preformed wire projection, with permanent lateral fixation of distal wire projection |
| | variation. |
| 7G- | Solid left perspective view, of general suture needle and activated protective non- |
| | preformed wire projection, with permanent lateral fixation of distal wire projection. |
| 7H- | Transparent front orthogonal view of activated wire projection, with permanent |
| | lateral fixation of distal wire projection. |
| 7I- | Transparent front orthogonal view of inactivated wire projection, with permanent |
| | lateral fixation of distal wire projection. |
| 7J- | Solid front orthogonal view of inactivated wire projection, with permanent lateral |
| | fixation of distal wire projection. |
| 7K- | Solid front orthogonal view of inactivated wire projection, with permanent lateral |
| | fixation of distal wire projection. |
| 7L- | Close up perspective view of projection wire loop. |
| 7M- | Cross-sectional side view of lateral secondary channel placement for wire location. |
| 7N- | Close-up view showing attachment to main slider or extension body. |
| 7O- | Lateral transparent close up view distal wire fixation point. |
| Element | |
| 7P- | Nesting channel for activated retracted projection wire. |
| 7Q- | Secondary Channels. |
| 7R- | Projection wire. |
| 7S- | Guide channel. |
| 7T- | Connection of projection loop to extension shaft or mechanism of action assembly. |

TABLE 8
Information on FIG. 8 set.

| | Information |
|---|---|
| FIG. | |
| 8- | Solid left perspective view, of general suture needle and |
| | inactivated protective Break away tip variation. |
| 8A- | Solid left perspective view, of general suture needle and |
| | activated protective Break away tip variation. |
| 8B- | Transparent front orthogonal view of activated break away tip. |
| 8C- | Transparent front orthogonal view of inactive break away tip. |
| 8D- | Transparent front silhouette view of extension and retraction |
| | sequence, of break away needle tip design. |
| 8E- | Solid front orthogonal view of activated break away tip. |
| 8F- | Solid front orthogonal view of inactivated break away tip. |
| Element | |
| 8G- | Close-up of main slider extension, direct SMA point, flexible |
| | member, and or other attachment configuration. |
| 8H- | Represents the break away, and intended free movement. |
| 8I- | Attachment point for flexible member extension within needle |
| | point. |
| 8J- | Flexible member extension. |
| 8K- | Flexible member attachment point to main slider extension. |
| 8L- | Break away tip main body. |
| 8M- | Blunted edge of main distal main needle body. |

TABLE 9
Information on FIG. 9 set.

| | Information |
|---|---|
| FIG. | |
| 9- | Properties of Shape memory alloy in relation to needle mechanism. |
| 9A- | Showing the SMA mechanism at rest and then activation. |
| 9B- | Showing the shape memory effect of the Shape Memory Alloy mechanism. |
| 9C- | Showing the activation and deactivation properties of the SMA mechanism. |
| 9D- | Showing the relation of the SMA mechanism in relation to the activation of the |
| | blunting mechanism. |
| 9E- | This vertical area denotes the location of the blunting mechanism either prior to or |
| | during activation of the particular mechanism. |
| 9F- | This vertical area denotes the location of the blunting mechanism either prior to or |
| | during activation of the particular mechanism. |
| 9G- | This vertical area shows the length of the SMA mechanism relative to the spring |
| | extension and or contraction of mechanism and its length during each phase. |
| Element | |
| 9H- | Inactive SMA with extension spring and mechanism properties. |
| 9I- | Activated SMA with extensions spring and properties. |
| 9J- | Deactivated SMA with extension spring and properties. |
| 9K- | Corresponds to 9H showing the relation of the mechanism to the blunted portion of |
| | the mechanism in relation to a general suture needle shape. |
| 9L- | Corresponds to 9I showing the relation of the mechanism to the blunted portion of |
| | the mechanism in relation to a general suture needle shape. |
| 9M- | Corresponds to 9J showing the relation of the mechanism to the blunted portion of |
| | the mechanism in relation to a general suture needle shape. |
| 9N- | SMA at inactive deformed state. |
| 9O- | SMA returning to reformed state upon activation. |
| 9P- | Corresponds to 9N showing the relation of the mechanism to the blunted portion of |
| | the mechanism in relation to a general suture needle shape. |
| 9Q- | Corresponds to 9N showing the relation of the mechanism to the blunted portion of |
| | the mechanism in relation to a general suture needle shape. |

TABLE 10
Information on FIG. 10 set.

| | Information |
|---|---|
| FIG. | |
| 10- | Broad figure of parts. |
| 10I- | Assembly in relation to needle body assembly (not all variations shown). |
| 10J- | Assembly in relation to needle body assembly (not all variations shown). |
| 10K- | Assembly in relation to needle body assembly (not all variations shown). |
| 10L- | SMA Shape memory application assembly in relation to needle body assembly |
| | (not all variations shown). |
| 10.1AA- | Break away one time use mechanism, SMA loop with retention crossbar variety. |
| 10.1AB- | Side view, break away one time use mechanism, SMA loop with retention crossbar |
| | variety, inactivated view (spring not shown). |
| 10.1AC- | Break away one time use mechanism, SMA loop with retention crossbar variety, |
| | inactivated side view (spring not shown). |
| 10.1AD- | Break away one time use mechanism, SMA loop with retention crossbar variety, |
| | activated side view (spring shown extended & divided for clarity). |
| 10.1B- | Break away one time use mechanism, SMA loop with crimped retention area. |
| 10.1BA- | Break away one time use crimped retention mechanism, inactivated. |
| 10.1BB- | Break away one time use crimped retention mechanism, side view, inactivated. |
| 10.1BC- | Break away one time use crimped retention mechanism, activated. |
| 10.1C- | Break away one time use mechanism with glued in SMA spring into retention area. |
| 10.1CA- | Break away one time use retention mechanism, with glued in SMA spring into |
| | retention area, inactivated. |
| 10.1CB- | Break away one time use retention mechanism, with glued in SMA spring into |
| | retention area, side view, inactivated. |
| 10.1CC- | Break away one time use retention mechanism, with glued in SMA spring into |
| | retention area, side view, activated. |
| 10.1D- | Break away one time use mechanism, with lateral SMA wire fixated with a vertical |
| | retention focus area to slider plate, inactivated. |
| 10.1DA- | Break away one time use mechanism, with lateral SMA wire fixated with a vertical |
| | retention focus area to slider plate, front view, inactivated. |
| 10.1DB- | Break away one time use mechanism, with lateral SMA wire fixated with a vertical |
| | retention focus area to slider plate, front view inactivated. |
| 10.1DC- | Break away one time use mechanism slider plate, inactivated, top view. |
| 10.1DD- | Break away one time use mechanism slider plate, activated, top view. |
| 10.1E- | One time use break away mechanism, with preformed SMA bend for retention. |
| 10.1EA- | One time use break away mechanism, with preformed SMA bend for retention, |
| | side view, activated. |
| 10.1EB- | One time use break away mechanism, with preformed SMA bend for retention, |
| | side view, activated (spring shown exploded, for clarity). |
| 10.1F- | One time use break away mechanism with SMA plate (series of views). |
| 10.1FA- | Side view of inactivated one time use break away mechanism with an SMA plate. |
| 10.1FB- | Top view of inactivated plate showing the retaining body, stopped. |
| 10.1FC- | Side view of activated one time use break away mechanism with an SMA plate. |
| 10.1FD- | top view of activated plate showing the opening of the plate upon activation. |
| Elements | |
| 10A- | SMA with variations and properties. (Not all shown). |
| 10B- | Proximal end connection point with variations and properties (not all shown). |
| 10C- | General extension spring (variations not shown). |
| 10D- | Denotes a connection point or connector plate |
| 10E- | Extension shaft or slider with variations (not all shown). |
| 10F- | Conduction wire with variations (not all shown). |
| 10G- | Denoted insulating layer. |
| 10H- | Shirt retraction spring, with variations (not all shown). |
| 10.1AE- | Crossbar. |
| 10.1AF- | Cross bar support. |
| 10.1AG- | Slider plate. |
| 10.1AH- | Crossbar aperture. |
| 10.1AI- | Extension spring. |
| 10.1BD- | Crimping area. |
| 10.1BE- | Slider plate. |
| 10.1CD- | Slider plate. |
| 10.1CE- | Retention area. |
| 10.1CF- | Focus of glued area. |
| 10.1DE- | Retention focus joint. |
| 10.1DF- | Retention focus joint, fixation depression. |
| 10.1DG- | Guide channel. |
| 10.1DH- | Slider plate with guide channel. |
| 10.1DI- | Extension spring. |
| 10.1EC- | Retention bend in SMA wire. |
| 10.1FE- | SMA plate. |
| 10.1FF- | Aperture. |
| 10.1FG- | Retention body. |
| 10.1FH- | Slider plate. |
| 10.1FI- | Extension spring applicable to this mechanism. |

TABLE 11
Information on FIG. 11 set.

| | Information |
|---|---|
| FIG. | |
| 11- | Side view of top and bottom connection point options. |
| 11A- | Top view of top bottom connection point option. |
| 11AA- | Cross sectional view of separated to bottom |
| | conduction point options. |
| 11B- | Side view of top and full needle body conduction point options. |
| 11C- | Top view of separated top bottom conduction point options. |
| 11CA- | Cross sectional view of separated top and full needle |
| | body conduction point options. |
| 11D- | Side view of 3 piece conduction point options. |
| 11E- | Top view of separated 3 piece conduction point option. |
| 11EA- | Cross sectional view of separated 3 piece |
| | conduction point option. |
| Element | |
| 11F- | Transverse cannula. |
| 11G- | Entire length top bottom |
| 11H- | Ring fixation with adhesive. |
| 11I- | Glue and teeth fixation. |
| 11J- | Glue only fixation. |
| 11K- | Other standard crimping fixation. |
| 11L- | Grasping teeth close-up cross-sectional view. |
| 11M- | Midline ring. |
| 11N- | Top plate. |
| 11O- | Bottom plate. |
| 11P- | Left side panel. |
| 11Q- | Right side panel. |
| 11R- | Contact, joining point, (not shown in all figures) |
| 11S- | Joining ring. |
| 11T- | Ring side view. |
| 11U- | Joining space. |
| 11V- | Joining teeth. |

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

Any appendix or appendices hereto are incorporated by reference as part of the specification and/or drawings.

Where the terms “comprise”, “comprises”, “comprised”, or “comprising” are used herein, they are to be interpreted as specifying the presence of the stated features, integers, steps, or components referred to, but not to preclude the presence or addition of one or more other feature, integer, step, component, or group thereof.

The invention has been described with reference to various specific and preferred embodiments and techniques. However, it should be understood that many variations and modifications may be made while remaining within the spirit and scope of the invention. It will be apparent to one of ordinary skill in the art that compositions, methods, devices, device elements, materials, procedures and techniques other than those specifically described herein can be applied to the practice of the invention as broadly disclosed herein without resort to undue experimentation. All art-known functional equivalents of compositions, methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example or illustration and not of limitation.

### REFERENCES

U.S. Pat. Nos. 5,437,681; 5,540,705; 5,728,108; 6,832,478 by Anderson, et al. issued Dec. 21, 2004; U.S. Pat. No. 5,947,983 by Solar, et al. issued Sep. 7, 1999; U.S. Pat. No. 6,641,593 by Schaller, et al. issued Nov. 4, 2003; U.S. Pat. No. 6,641,564 by Kraus issued Nov. 4, 2003; 20040068242 by McGuckin, James F. JR. published Apr. 8, 2004; U.S. Pat. No. 4,926,860 by Stice, et al. issued May 22, 1990.

US patent documents:

| Patent | | Issue/Publication |
|---|---|---|
| Document No. | Inventor | Date |
| 269,468 | Rounds | Dec. 19, 1882 |
| 1,327,577 | Turner | 6-Jan-1920 |
| 1,689,889 | Nunes | 24-Feb-1927 |
| 1,844,364 | Lowrie | 12-Jan-1931 |
| 2,581,114 | Larson | 13-Apr-1950 |
| 2,706,987 | Bramstedt | 12-Oct-1952 |
| 2,928,395 | Forbes et al. | 20-Jun-1957 |
| 2,936,759 | Yuhas | 3-Oct-1957 |
| 3,212,502 | Myers | 25-Jan-1963 |
| 3,608,095 | Barry | 28-Sep-1971 |
| 3,608,539 | Miller | 28-Sep-1971 |
| 3,735,765 | Ichelson | 29-May-1973 |
| 3,762,418 | Wasson | 2-Oct-1973 |
| 3,892,240 | Park | 17-Jul-1975 |
| 3,910,280 | Talonn | 7-Oct-1975 |
| 3,913,586 | Baumgarten | 21-Oct-1975 |
| 3,914,801 | Dick et al. | 28-Oct-1975 |
| 3,993,076 | Forgarty | 23-Nov-1976 |
| 4,074,718 | Morrison Jr. | 21-Feb-1978 |
| 4,133,339 | Naslund | 9-Jan-1979 |
| 4,140,125 | Smith | 20-Feb-1979 |
| 4,392,495 | Bayers | 12-Jul-1983 |
| 4,527,564 | Eguchi et al. | 9-Jul-1985 |
| 4,602,636 | Noiles | 29-Jul-1986 |
| 4,672,734 | Kawanda et al. | 16-Jun-1987 |
| 4,722,384 | Matsutani | 2-Feb-1988 |
| 4,777,096 | Borysko | 11-Oct-1988 |
| 4,799,311 | Matsutani | 24-Jan-1989 |
| 4,805,292 | Noguchi | 21-Feb-1989 |
| 4,821,717 | Wehrli | 18-Apr-1989 |
| 4,890,614 | Kawanda et al. | 2-Jan-1990 |
| 4,899,746 | Brunk | 13-Feb-1990 |
| 4,901,722 | Noguchi | 20-Feb-1990 |
| 4,932,962 | Yoon et al. | 12-Jun-1990 |
| 4,966,143 | Meinershagen | 30-Oct-1990 |
| 5,002,563 | Pyka et al. | 26-Mar-1991 |
| 5,012,066 | Matsutani et al. | 30-Apr-1991 |
| 5,024,323 | Bolton | 18-Jun-1991 |
| 5,100,432 | Matsutani | 31-Mar-1992 |
| 5,102,418 | Granger et al. | 7-Apr-1992 |
| 5,123,910 | McIntosh | 23-Jun-1992 |
| 5,123,911 | Granger et al. | 23-Jun-1992 |
| 5,155,943 | Matsutani et al. | 20-Oct-1992 |
| 5,168,619 | Proto et al. | 8-Dec-1992 |
| 5,178,628 | Otsuka et al. | 12-Jan-1993 |
| 5,180,053 | Cascie et al. | 19-Jan-1993 |
| 5,180,354 | Jones | 19-Jan-1993 |
| 5,180,385 | Sontag | 19-Jan-1993 |
| 5,192,483 | Kilgrow et al. | 9-Mar-1993 |
| 5,219,358 | Bendel et al. | 15-Jun-1993 |
| 5,222,962 | Burkhart | 29-Jun-1993 |
| 5,230,352 | Putnam | 27-Jul-1993 |
| 5,236,443 | Sontag | 17-Aug-1993 |
| 5,242,458 | Bendel et al. | 7-Oct-1993 |
| 5,269,806 | Sardelis et al. | 14-Dec-1993 |
| 5,431,678 | Rogers | 11-Jul-1995 |
| 5,439,469 | Heaven et al. | 8-Aug-1995 |
| 5,468,251 | Buelna | 21-Nov-1995 |
| 5,533,982 | Rizk et al. | 9-Jul-1996 |
| 5,542,434 | Imran et al. | 6-Aug-1996 |
| 5,549,619 | Peters et al. | 27-Aug-1996 |
| 5,549,629 | Thomas et al. | 27-Aug-1996 |
| 5,556,410 | Mittermeir et al. | 17-Sep-1996 |
| 5,562,678 | Booker | 8-Oct-1996 |
| 5,569,271 | Hoel | 29-Oct-1996 |
| 5,597,378 | Jervis | 28-Jan-1997 |
| 5,632,752 | Buelna | 27-May-1997 |
| 5,693,072 | McIntosh | 2-Dec-1997 |
| 5,741,299 | Rudt | 21-Apr-1998 |
| 5,782,896 | Chen et al. | 21-Jul-1998 |
| 5,810,851 | Yoon et al. | 22-Sep-1998 |
| 5,833,697 | Ludwick | 10-Nov-1998 |
| 5,853,432 | Etzbach et al. | 29-Dec-1998 |
| 5,895,395 | Yeung | 20-Apr-1999 |
| 5,902,320 | Matsutani et al. | 11-May-1999 |
| 5,904,692 | Steckel et al. | 18-May-1999 |
| 5,928,268 | Butwell et al. | 27-Jul-1999 |
| 5,941,899 | Granger et al. | 24-Aug-1999 |
| 6,010,514 | Burney et al. | 4-Jan-2000 |
| 6,056,771 | Proto | 2-May-2000 |
| 6,143,004 | Davis et al. | 7-Nov-2000 |
| 6,159,223 | Danks et al. | 12-Dec-2000 |
| 6,190,332 | Muni et al. | 20-Feb-2001 |
| 6,214,030 | Matsutani et al. | 10-Apr-2001 |
| 6,346,111 | Gordon et al. | 12-Feb-2002 |
| 6,375,628 | Zadne-Azizi et al. | 23-Apr-2002 |
| 6,425,887 | McGuckin et al. | 30-Jul-2002 |
| 6,514,263 | Stefanchik et al. | 4-Feb-2003 |
| 6,592,559 | Pakter et al. | 15-Jul-2003 |
| 6,626,973 | Park | 30-Sep-2003 |
| 2003/0120308 | Thierry et al. | 26-Jun-2003 |

1. A suture needle assembly, comprising a suture needle and an activatable sheath.

2. The suture needle assembly of claim 1 wherein said sheath is activatable by electricity.

3. The suture needle assembly of claim 1 wherein said sheath is activatable by heat.

4. The suture needle assembly of claim 1 wherein said needle comprises a first tissue penetration end and a second end for suture attachment, an external casing and an internal compartment, an outer surface along a longitudinal axis of said external casing, a sheath activator disposed inside said casing and electrically or thermally responsive to said casing, and said activatable sheath operatively connected to said sheath activator; wherein a portion of said sheath is capable of reaching a protective position proximal to said first end of said needle.

5. The suture needle assembly of claim 4 wherein said sheath activator comprises a shape memory alloy component.

6. The suture needle assembly of claim 5 wherein said shape memory alloy component is nitinol.

7. The suture needle of claim 4 wherein said sheath further comprises a catch point, notch, or securing means for maintaining said sheath in an activated position upon activation.

8. The suture needle assembly of claim 4 wherein said sheath activator comprises a shape memory plastic component.

9. The suture needle assembly of claim 4 wherein said sheath comprises a shape memory alloy or shape memory plastic.

10. The suture needle assembly of claim 4 further comprising an initiator of said sheath activator, wherein said activator is capable of receiving energetic exposure from said initiator.

11. The suture needle assembly of claim 10 wherein said initiator comprises a source of heat or electricity.

12. The suture needle assembly of claim 4 wherein said activatable sheath and said activator are unitary.

13. The suture needle assembly of claim 4 further comprising an extension shaft connected at a first end to said activator and connected at a second end to said sheath.

14. The suture needle assembly of claim 1 wherein said activatable sheath is reversibly capable of activation.

15. The suture needle assembly of claim 14 wherein said reversibly activatable sheath is provided in an initially armed configuration.

16. The suture needle assembly of claim 1 wherein said activatable sheath comprises a cylindrical projection.

17. The suture needle assembly of claim 1 wherein said activatable sheath comprises a hood-shaped shield.

18. The suture needle assembly of claim 1 wherein said activatable sheath comprises a cylindrical projection.

19. The suture needle assembly of claim 1 wherein said activatable sheath comprises a loop.

20. The suture needle assembly of claim 1 wherein said needle comprises a sheath receiving aperture.

21. The suture needle assembly of claim 20 wherein said sheath receiving aperture is a groove or notch.

22. The suture needle assembly of claim 1 wherein said activatable sheath is capable of assuming a retracted position that is substantially flush along a needle outer surface.

23. A suturing apparatus comprising a suture needle assembly of any of claims 1-22 and a needle holder.

24. A suturing apparatus comprising a suture needle assembly of claim 4 and a modified needle holder; wherein the modified needle holder comprises a first holding tip and a second holding tip, and the first and second holding tips are energetically connected to an energy source; and wherein the first holding tip and second holding tip are capable of contacting a first and a second needle assembly energy contact surface, wherein said contact surfaces are energetically contacted to said activator of said needle assembly, and wherein said holding tips are capable of delivering energy to said contact surfaces.

25. The suturing apparatus of claim 24 wherein said energy source is electricity.

26. The suturing apparatus of claim 24 wherein said energy source is heat.

27. The needle assembly of claim 4 wherein said external casing comprises a first outer surface portion and a second outer surface portion capable of forming an electrical circuit.

28. A method of reducing a probability of an accidental suture needle puncture event comprising; providing a safety suture needle of claim 1, performing a suturing procedure, activating an electrically or thermally responsive sheathing mechanism of said needle so as to move a sheath of said needle into a protective position with respect to a sharp tip of said needle; thereby reducing a probability of an accidental suture needle puncture event.

29. A method of suturing, comprising providing a safety suture needle having a shape memory alloy for selectively sheilding a portion of the needle, and performing a suturing procedure with said safety suture needle.

30. A modified needle holder, comprising a needle holder having a first holding tip and a second holding tip, each electrically connected to a power source and capable of delivering electricity.

31. The modified needle holder of claim 30, further comprising a control switch or means for circuit regulation.

32. A suture needle comprising a needle body, a flexible extension member and a needle distal tip, the distal tip being attached to the needle body by the flexible extension member.

33. The suture needle of claim 32, wherein the flexible extension member is configured to selectively allow the distal tip to pivot with respect to the needle body.

34. A suturing kit comprising the suture needle assembly of claim 1,
a modified needle holder comprising a needle holder having a first holding tip and a second holding tip, each electrically connected to a power source and capable of delivering electricity,
and suture material.

35. A suture needle comprising a unifying element, wherein the unifying element is either continuously connected to a needle casing or connected at multiple points thereto, wherein said unifying element comprises a shape memory material component and is capable upon fracturing event of said needle casing of preventing dissociation of a needle part from another needle part or the remainder of the needle body.

36. The suture needle of claim 35 wherein the shape memory material component is nitinol.
